(19) United States
(12) Patent Application Publication
(10) Pub. No.: US 2020/0330367 A1
(43) Pub. Date: Oct. 22, 2020

(54) STEM CELL COMPOSITIONS FOR COSMETIC AND DERMATOLOGIC USE

(71) Applicant: NeoStem Oncology, LLC, Irvine, CA (US)

(72) Inventors: Gabriel Nistor, Laguna Niguel, CA (US); Aleksandra J. Poole, San Marcos, CA (US); Hans S. Kierstead, Laguna Beach, CA (US)

(21) Appl. No.: 16/869,394

(22) Filed: May 7, 2020

Related U.S. Application Data
(63) Continuation of application No. 15/565,090, filed on Oct. 6, 2017.
(60) Provisional application No. 62/145,345, filed on Apr. 9, 2015.

(51) Int. Cl. A61K 8/98; A61Q 19/08; C12N 5/00; A61K 45/06; A61K 35/545; A61K 38/17; A61K 8/06; A61K 8/34; A61K 8/64; A61Q 19/00

(52) U.S. Cl. CPC A61K 8/981; A61Q 19/08; C12N 5/00; A61K 45/06; A61K 35/545; C12N 2502/02; A61K 8/068; A61K 8/345; A61K 8/64; A61Q 19/00; A61K 38/1741

(57) ABSTRACT

Disclosed herein are cosmetic compositions containing stem cell secreted proteins that, in a cosmetic amount, are effective to enhance appearance of photo-damaged, or aged, skin.

### CROSS-REFERENCE TO RELATED APPLICATIONS

The present application is a continuation of U.S. application Ser. No. 15/565,090, filed Oct. 6, 2017, which is a 35 U.S.C. 371 national phase entry of PCT/US2016/026968, filed Apr. 11, 2016, which claims the benefit under 35 U.S.C. 119(e) to U.S. Provisional Patent Application 62/145,345 filed Apr. 9, 2015 and each of which is incorporated by reference herein in its entirety.

### FIELD

The present disclosure relates to stem cell secreted proteins for use in cosmetic and dermatologic compositions to improve the appearance of the skin.

### BACKGROUND

The skin is the largest organ in the body consisting of several layers and plays an important role in biologic homeostasis. The epidermis, which is composed of several layers beginning with the stratum corneum, is the outermost layer of the skin. The innermost skin layer is the deep dermis. The skin has multiple functions, including thermal regulation, metabolic function (vitamin D metabolism), and immune functions.

In humans, the usual thickness of the skin is from 1-2 mm, although there is considerable variation in different parts of the body. The relative proportions of the epidermis and dermis also vary, and a thick skin is found in regions where there is a thickening of either or both layers.

The entire skin surface is traversed by numerous fine furrows, which run in definite directions and cross each other to bound small rhomboid or rectangular fields. These furrows correspond to similar ones on the surface of the dermis so that, in section, the boundary line between epidermis and dermis appears wavy.

The epidermis provides body's buffer zone against the environment. It provides protection from trauma, excludes toxins and microbial organisms, and provides a semi-permeable membrane, keeping vital body fluids within the protective envelope. Traditionally, the epidermis has been divided into several layers, of which two represent the most significant ones physiologically. The basal-cell layer, or germinative layer, is of importance because it is the primary source of regenerative cells. In the process of wound healing, this is the area that undergoes mitosis in most instances. The upper epidermis, including stratum and granular layer, is the other area of formation of the normal epidermal-barrier function.

Stratum corneum is an avascular, multilayer structure that functions as a barrier to the environment and prevents transepidermal water loss. Recent studies have shown that enzymatic activity is involved in the formation of an acid mantle in the stratum corneum. Together, the acid mantle and stratum corneum make the skin less permeable to water and other polar compounds, and indirectly protect the skin from invasion by microorganisms. Normal surface skin pH is between 4 and 6.5 in healthy people; it varies according to area of skin on the body. This low pH forms an acid mantle that enhances the skin barrier function.

Other layers of the epidermis below the stratum corneum include the stratum lucidum, stratum granulosum, stratum germinativum, and stratum basale. Each contains living cells with specialized functions. For example melanin, which is produced by melanocytes in the epidermis, is responsible for the color of the skin. Langerhans cells are involved in immune processing.

The basement membrane both separates and connects the epidermis and dermis. When epidermal cells in the basement membrane divide, one cell remains, and the other migrates through the granular layer to the surface stratum corneum. At the surface, the cell dies and forms keratin. Dry keratin on the surface is called scale. Hyperkeratosis (thickened layers of keratin) is found often on the heels and indicates loss of sebaceous gland and sweat gland functions if the patient is diabetic. The basement membrane atrophies with aging; separation between the basement membrane and dermis is one cause for skin tears in the elderly.

The dermis, or the true skin, is a vascular structure that supports and nourishes the epidermis. In addition, there are sensory nerve endings in the dermis that transmit signals regarding pain, pressure, heat, and cold. The dermis is divided into two layers: the superficial dermis consists of extracellular matrix (collagen, elastin, and ground substances) and contains blood vessels, lymphatics, epithelial cells, connective tissue, muscle, fat, and nerve tissue. The vascular supply of the dermis is responsible for nourishing the epidermis and regulating body temperature. Fibroblasts are responsible for producing the collagen and elastin components of the skin that give it turgor. Fibronectin and hyaluronic acid are secreted by the fibroblasts. The structural integrity of the dermis plays a role in the normal function and youthful appearance of the skin.

The deep dermis is located over the subcutaneous fat; it contains larger networks of blood vessels and collagen fibers to provide tensile strength. It also consists of fibroelastic connective tissue, which is yellow and composed mainly of collagen. Fibroblasts are also present in this tissue layer. The well-vascularized dermis withstands pressure for longer periods of time than subcutaneous tissue or muscle. The collagen in the skin gives the skin its toughness.

Substances are applied to the skin to elicit one or more of four general effects: an effect on the skin surface, an effect within the stratum corneum; an effect requiring penetration into the epidermis and dermis; or a systemic effect resulting from delivery of sufficient amounts of a given substance through the epidermis and the dermis to the vasculature to produce therapeutic systemic concentrations. One example of an effect on the skin surface is formation of a film. Film formation may be protective (e.g., sunscreen) and/or occlusive (e.g., to provide a moisturizing effect by diminishing loss of moisture from the skin surface). One example of an effect within the stratum corneum is skin moisturization, which may involve the hydration of dry outer cells by surface films or the intercalation of water in the lipid-rich intercellular laminae. The stratum corneum also may serve as a reservoir phase or depot wherein topically applied substances accumulate due to partitioning into, or binding with, skin components.

It generally is recognized that short-term penetration occurs through the hair follicles and the sebaceous apparatus of the skin, while long term penetration occurs across cells. Penetration of a substance into the viable epidermis and dermis may be difficult to achieve, but once it has occurred, the continued diffusion of the substance into the dermis is likely to result in its transfer into the microcirculation of the dermis and then into the general circulation. It is possible, however, to formulate delivery systems that provide substantial localized delivery.

Percutaneous absorption is the absorption of substances from outside the skin to positions beneath the skin, including into the blood stream. The epidermis of human skin is highly relevant to absorption rates. Passage through the stratum corneum marks the rate-limiting step for percutaneous absorption. The major steps involved in percutaneous absorption of, for example, a drug include the establishment of a concentration gradient, which provides a driving force for drug movement across the skin, the release of drug from the vehicle into the skin-partition coefficient and drug diffusion across the layers of the skin-diffusion coefficient.

There are many factors that affect the rate of percutaneous absorption of a substance. Primarily they are as follows: (i) Concentration. The more concentrated the substance, the greater the absorption rate. (ii) Size of skin surface area. The wider the contact area of the skin to which the substance is applied, the greater the absorption rate. (iii) Anatomical site of application. Skin varies in thickness in different areas of the body. A thicker and more intact stratum corneum decreases the rate of absorbency of a substance. The stratum corneum of the facial area is much thinner than, for example, the skin of the palms of the hands. The facial skin's construction and the thinness of the stratum corneum provide an area of the body that is optimized for percutaneous absorption to allow delivery of active agents both locally and systemically through the body. (iv) Hydration. Hydration (meaning increasing the water content of the skin) causes the stratum corneum to swell which increases permeability. (v) Skin temperature. Increased skin temperature increases permeability. (vi) Composition. The composition of the compound and of the vehicle also determines the absorbency of a substance.

Most substances applied topically are incorporated into bases or vehicles. The vehicle chosen for a topical application will greatly influence absorption, and may itself have a beneficial effect on the skin. Factors that determine the choice of vehicle and the transfer rate across the skin are the substance's partition coefficient, molecular weight and water solubility. The protein portion of the stratum corneum is most permeable to water soluble substances and the lipid portion of the stratum corneum is most permeable to lipid soluble substances. It follows that substances having both lipid and aqueous solubility may traverse the stratum corneum more readily.

Particle size and rheology (meaning flow characteristics) often are key indicators of a cosmetic product's final performance. Liposomes often are used when formulating a moisturizing product, because moisturizing products need to rapidly absorb into the skin. Such particles generally measure less than about 200 nm.

Pluripotent stem cells are characterized by the ability to self-replicate and differentiate. Stem cells are characterized typically by morphology as well as the presence of characteristic markers. For example, morphology of a stem cell is typically dense, well delimited small cells with a large nucleus representing about 80 to 95% of the total cellular volume. Stem cell differentiation can result in a phenotypic change—the most commonly observed change is in cell morphology. For example, the proportion of nucleus to cytoplasm is reduced, cells acquire migratory capability, and the colony edges become less defined. Stem cell differentiation can also result in a loss of stem cell markers (e.g., OCT4, SSEA4, TRA1-81) or telomerase activity. Stem cell differentiation can further result in acquiring markers or morphologies characteristic of one or more of the three embryonic germ layers—ectoderm, mesoderm or endoderm. Under certain conditions, stem cells can grow outside of stem cell colonies and their number and the growth can be determined by immunolabeling with markers characteristic of stem cells.

Undifferentiated stem cells contain a strong replicative apparatus. While protein synthesis is parsimonious during self-renewal, differentiation induces an anabolic switch, with global increases in transcript abundance, polysome content, protein synthesis, and protein content.

Spontaneous differentiation of stem cells is normal and reflects normal functioning stem cells. Spontaneous differentiation results in a cellular mass—stroma—which fills the space between the colonies. The proportion between the stroma representing differentiated cells and colonies representing non-differentiated cells can vary, as long as the stem cell colonies are properly defined (delimitation, dense, typical cellular content). Stem cells can range from a single colony in a culture dish (which can be 0.1% of the total cell number) to virtually 100% with a complete absence of stromal cells. The proportion between stroma (differentiated cells) and colonies (stem cells) in media can be regulated by other factors unrelated to the media composition, for example the ratio that cells are split when passaged.

Current methods to propagate undifferentiated pluripotent stem cells (e.g., embryonic stem cells, induced pluripotent stem (iPS) cells) use culture reagents that eliminate differentiated cells and promote expansion of undifferentiated cells.

Such reagents typically contain high concentrations of specific growth factors (e.g., FGF, TGFβ) and lack differentiating factors, such as bone morphogenic proteins (e.g., BMP2, BMP4). Other factors, including G-protein coupled receptor (GPCR) ligands (e.g., hormones) and integrin ligands (e.g., laminin, collagen) are also responsible for the maintenance of pluripotency and used in typical stem cell culture systems.

Media formulations to expand human embryonic stem cells (hESC) for multiple passages and maintenance of pluripotency and normal karyotype have been described. U.S. Pat. No. 7,977,096 (incorporated by reference herein for all it discloses regarding stem cell media) describes a chemically defined media that can maintain self-renewal and pluripotency of pluripotent stem cells for many passages. By manipulating the hyaluronan/hyaluronidase system, the tendency to differentiate towards endoderm or ectoderm (“biasing”) can be manipulated.

### SUMMARY

Disclosed herein are compositions and methods resulting from partial differentiation of pluripotent stem cells which maintaining a high rate of self-renewal. Protein secretions of the resulting stem cell population and the beneficial use of these secretions in cosmetic applications are disclosed.

Thus, disclosed herein are cosmetic or dermatologic compositions comprising: a culture media collected from a culture comprising a population of biased pluripotent stem cells being characterized by expression of stem cell markers OCT4 and SSEA4 without substantial differentiation; wherein the culture media comprises secretory products of the population of biased pluripotent stem cells and human fetuin; at least one cosmetically or dermatologically acceptable carrier, wherein an effective amount of the cosmetic or dermatologic composition is effective to enhance appearance of skin. In certain embodiments, the fetuin is secreted by the biased pluripotent stem cells.

The cosmetic or dermatologic composition according to claim 1, wherein the effective amount of the composition is effective to enhance appearance of skin by improving one or more of tactile roughness, visual softness; light reflected radiance; appearance of lines/wrinkles, skin tone; skin clarity, redness; firmness/elasticity, radiance, skin texture/smoothness, or overall appearance.

The cosmetic or dermatologic composition according to claim 1, wherein the effective amount of the secretory product of the population of biased pluripotent stem cells is effective to modulate one or more of proliferation, inflammation, angiogenesis or apoptosis of epidermal cells.

In certain embodiments, the secretory product of the population of biased pluripotent stem cells includes: an effective amount of an extracellular matrix factor secreted by the biased cell culture into the culture media; or an effective amount of a growth factor secreted by the biased cell culture into the culture media; or a combination thereof.

In some embodiments, the effective amount of the growth factor: has a stimulatory effect on cell proliferation; or has an inhibitory effect on cell proliferation; or has an anti-apoptotic effect on cells; or has a vasculogenic effect; or a combination thereof. In some embodiments, the growth factor is a cytokine.

In certain embodiments, an effective amount of the cytokine: has an inhibitory effect on the immune system; or has a stimulatory effect on the immune system; or a combination thereof.

In some embodiments, the secretory product of the population of biased pluripotent stem cells includes: an effective amount of a proteolytic enzyme secreted by the biased cell culture into the culture media; or an effective amount of an enzyme inhibitor secreted by the biased cell culture into the culture media; or a combination thereof.

In some embodiments, the population of biased pluripotent stem cells is of human origin. In some embodiments, the population of biased pluripotent stem cells comprises a population of induced pluripotent stem (iPS) cells; a population of embryonic stem (ES) cells; a population of germinal cells; tissue-specific stem cells; or a population of adult stem cells.

In certain embodiments, the composition comprises an effective amount of at least one further ingredient selected from a hydrophobic component; an emulsifier, a water-soluble humectant; a viscosifying agent; a ultraviolet absorbing agent; or an additional skin active agent. In some embodiments, the composition further comprises pentylene glycol. In some embodiments, the composition is in form of a microemulsion. In some embodiments, an effective amount of the hydrophobic component is effective to condition skin. In some embodiments, an effective amount of the water-soluble humectant is effective to promote water retention in the skin.

In certain embodiments, the skin is photo-damaged skin. In some embodiments, the skin is aged skin. In some embodiments, the skin is aged by intrinsic factors or extrinsic factors.

In certain embodiments, the culture media is present in the cosmetic composition at a concentration of about 1% to about 25%. In some embodiments, the culture media is present in the cosmetic composition at a concentration of 1%, 2%, 3%, 4%, 5%, 10%, 15%, 20%, or 25%.

Also disclosed herein are cosmetic or dermatologic compositions comprising: an effective amount of human fetuin and at least one cosmetically or dermatologically acceptable carrier, wherein an effective amount of the cosmetic or dermatologic composition is effective to enhance appearance of skin.

In certain embodiments, the effective amount of the composition is effective to enhance appearance of skin by improving one or more of tactile roughness, visual softness; light reflected radiance; appearance of lines/wrinkles, skin tone; skin clarity, redness; firmness/elasticity, radiance, skin texture/smoothness, or overall appearance. In some embodiments, the effective amount of composition is effective to modulate one or more of proliferation, inflammation, angiogenesis or apoptosis of epidermal cells.

In certain embodiments, the composition comprises an effective amount of at least one further ingredient selected from a hydrophobic component; an emulsifier, a water-soluble humectant; a viscosifying agent; a ultraviolet absorbing agent; or an additional skin active agent. In some embodiments, the composition further comprises pentylene glycol. In some embodiments, the composition is in form of a microemulsion. In some embodiments, an effective amount of the hydrophobic component is effective to condition skin. In some embodiments, an effective amount of the water-soluble humectant is effective to promote water retention in the skin.

In certain embodiments, the skin is photo-damaged skin. In some embodiments, the skin is aged skin. In some embodiments, the skin is aged by intrinsic factors or extrinsic factors.

In certain embodiments, the fetuin is present in the composition at a concentration of about 0.001 μg/ml to about 1 μg/ml. In some embodiments, the fetuin is present in the composition at a concentration of about 0.005 μg/ml.

Also provided herein are methods for enhancing appearance of skin, comprising providing a cosmetic or dermatologic composition disclosed herein; applying an effective amount of the composition topically; and improving one or more of tactile roughness, visual softness; light reflected radiance; appearance of lines/wrinkles, skin tone; skin clarity, redness; firmness/elasticity, radiance, skin texture/smoothness, or overall appearance.

### BRIEF DESCRIPTION OF THE DRAWINGS

FIG. 1 shows an exemplary system for collection of supernatant media.

FIG. 2A shows measured parameter changes with application of a sample preparation incorporating 5% culture media collected from a culture comprising a population of biased pluripotent stem cells.

FIG. 2B shows overall skin improvement with percent change from baseline with application of a sample preparation incorporating 5% culture media collected from a culture comprising a population of biased pluripotent stem cells.

FIG. 3A shows measured parameter changes with application of a sample preparation incorporating 25% culture media collected from a culture comprising a population of biased pluripotent stem cells.

FIG. 3B shows overall skin improvement with percent change from baseline with application of a sample preparation incorporating 25% culture media collected from a culture comprising a population of biased pluripotent stem cells.

FIG. 4 shows hematoxylin and eosin histology staining of skin biopsy from control subjects and subjects treated with a sample preparation incorporating 5% culture media collected from a culture comprising a population of biased pluripotent stem cells.

FIGS. 5A-B shows a histological comparison of biopsies from subjects treated with a sample preparation incorporating 5% (5A) or 25% (5B) culture media collected from a culture comprising a population of biased pluripotent stem cells and matched controls showing a significant increase in rete peg presence (p=0.039).

FIG. 6 shows representative images of filaggrin immunocytochemistry. The positive staining (more intense/darker coloration) shows increased intensity in the treatment sample.

FIGS. 7A-B shows a histological comparison of biopsies from subjects treated with a sample preparation incorporating 25% (7A) or 5% (7B) culture media collected from a culture comprising a population of biased pluripotent stem cells and matched controls showing a significant increase filaggrin positive area (p<0.001).

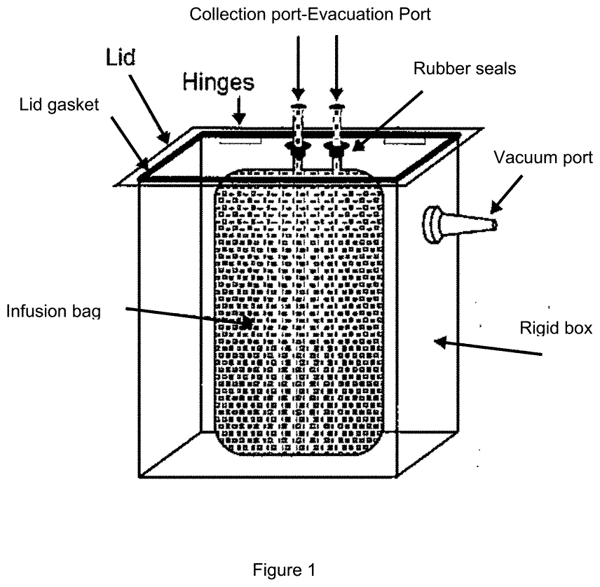

### DETAILED DESCRIPTION

Disclosed herein are compositions and methods resulting from partial differentiation of pluripotent stem cells which maintaining a high rate of self-renewal. Protein secretions of the resulting stem cell population and the beneficial use of these secretions in cosmetic applications are disclosed.

### Glossary

The term “active” refers to the ingredient, component or constituent of the disclosed compositions responsible for the intended cosmetic effect.

The term “aging skin” as used herein refers to the problem of exposed areas of the skin, such as the face, having the appearance of older skin far earlier than never exposed sites of the body.

As used herein, the term “angiogenesis” refers to the process of formation and development of blood vessels.

As used herein, the terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

The term “biased pluripotent stem cells” as used herein refers to a population of partially differentiated stem cells that retain the major markers for stemness (OCT4, SSEA4), but change metabolism with a global increase in transcription and protein synthesis. The biasing in generic culture conditions continues differentiation towards endoderm, mesoderm, or ectoderm lineages.

The term “bound” or any of its grammatical forms as used herein refers to the capacity to hold onto, attract, interact with, or combine with.

The term “cell culture” as used herein refers to a population of cells whose cell viability is maintained or sustained for at least a period of time in vitro. Not all cells are required to survive or proliferate in a disclosed complete media formulation and, in fact a small or even a large number of cells may die or senesce. Likewise, not all cells of a given cell culture are required to survive or proliferate in a disclosed complete media formulation.

The term “components” as used herein refers to particular compounds or ingredients that are present or make up a media formulation. Such components can be used in the media to sustain or maintain cell survival, viability or proliferation. Such components can be unrelated to cell survival, viability or proliferation, but may serve another purpose, such as a preservative, dye or coloring agent (e.g., to indicate pH of the media).

The terms “formulation” and “composition” are used interchangeably herein to refer to a product disclosed herein that comprises all active and inert ingredients.

The term “cosmetic” generally refers to (i) a substance intended to be rubbed, poured, sprinkled or sprayed on, introduced, or otherwise applied to the human body or other animal body or any part thereof for cleansing, beautifying, promoting attractiveness, or altering the appearance, and (ii) a substance intended for use as a component of any such substances, except that such term shall not include soap. Non-limiting examples of products included in this definition are skin moisturizers, eye and facial preparations, skin lighteners, masques, toners, and any material intended for use as a component of a skin cosmetic product. Generally, such products may be applied to large surface areas of skin, to damaged skin, and to delicate areas of the skin, and are left on the skin for long periods of time. A cosmetic may be employed, for example to counteract the visible effects of aging skin, environmental stresses, to maintain the skin and hair at its best, and to promote homeostasis (adaptability).

The term “cosmetically acceptable carrier” as used herein refers to a substantially non-toxic carrier conventionally usable for the topical administration of cosmetics, with which compounds will remain stable and bioavailable.

The term “cosmetic agent” as used herein refers to a factor, molecule, nucleic acid, protein, or other substance that provides a cosmetic effect.

The term “cosmetic amount” or amount cosmetically effective as used herein refers to an amount of a cosmetic agent that is sufficient to provide the intended benefit of treatment.

The term “cosmetic effect” as used herein refers to a consequence of treatment, the results of which are judged to be desirable and beneficial.

The term “cytokine” as used herein refers to small soluble protein substances secreted by cells which have a variety of effects on other cells. Cytokines mediate many physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “differentiation” as used herein refers to the process of development with an increase in the level of organization or complexity of a cell or tissue, accompanied with a more specialized function.

The term “embryonic stem cell,” “ES,” or “ESC” as used herein refers to primitive (undifferentiated) cells derived from a preimplantation-stage embryo that are capable of dividing without differentiating for prolonged period in culture, and are known to develop into cells and tissues of the three primary germ layers.

The term “extracellular matrix” as used herein refers to a scaffold in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fribronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid).

The term “fibroblast growth factor” (FGF) as used herein refers to a family of cytokines that possess broad mitogenic and angiogenic activities. To date, the FGF superfamily consists of 23 members, all of which contain a conserved 120 amino acid (aa) core region that contains six identical, interspersed amino acids. The superfamily members act extracellularly through four tyrosine kinase FGF receptors, with multiple specificities noted for almost all FGFs, which likely accounts for similar effects generated by many FGF molecules on common cell types. The FGFs, partly by way of their originally recognized proliferative activities, are considered to play substantial roles in development, angiogenesis, hematopoiesis, and tumorigenesis. Human FGF-1 (also known as FGF acidic, FGFa, ECGF and HBGF-1) is a 17-18 kDa non-glycosylated polypeptide that is expressed by a variety of cells from all three germ layers. Human FGF-2, otherwise known as FGF basic, HBGF-2, and EDGF, is an 18 kDa, non-glycosylated polypeptide that shows both intracellular and extracellular activity.

The term “growth” as used herein refers to a process of becoming larger, longer, or more numerous, or an increase in size, number, or volume.

The term “inflammatory mediators” or “inflammatory cytokines” as used herein refers to the molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, follistatin-like protein 1 (FSTL-1) and neuropeptides, proinflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IF-γ), and interleukin-12 (IL-12).

Among the pro-inflammatory mediators, IL-1, IL-6, and TNF-α are known to activate hepatocytes in an acute phase response to synthesize acute-phase proteins that activate complement. IL-1, IL-6, and TNF-α also activate bone marrow endothelium to mobilize neutrophils, and function as endogenous pyrogens, raising body temperature, which helps eliminating infections from the body.

Anti-inflammatory mediators include, without limitation, IL-1 receptor antagonist, IL-4, IL-6, II-10, IL-11, IL-13, IL-23 and TGFβ.

IL-6 has both pro- and anti-inflammatory properties. The IL-6 family of cytokines includes IL-6, IL-11, leukemia inhibitory factor (LIF), oncostatin M (OSM), ciliary inhibitory factor (CNTF), cardiotropin-1 (CT-1), cardiotrophin-like related cytokine and stimulating neurotrophin-1/B-cell stimulating factor 3 (NNT-1), neuropoietin (NPN), IL-27 and IL-31.

Fetuin-A, a hepatokine, is a pleotropic molecule with diverse (sometimes even contradictory) effects in different systems, brought about by interaction with a variety of receptors, including the insulin, transforming growth factor-β, and a plethora of Toll-like receptors (TLRs). As a pro-inflammatory molecule, fetuin-A contributes to insulin resistance and is an important link between liver, adipose tissue, and muscles. As an anti-inflammatory molecule, it plays an important anti-inflammatory role in sepsis and autoimmune disorders.

The term “induced pluripotent stem cells” or “iPSCs” as used herein refers to a type of pluripotent stem cell, similar to an embryonic stem cell, formed by the introduction of certain embryonic genes into a somatic cell (meaning any body cell other than gametes (egg or sperm); sometimes referred to as “adult” cells). Human iPSCs express stem cell markers and are capable of generating cells characteristic of all three germ layers.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “inhibitor” as used herein refers to a second molecule that binds to a first molecule thereby decreasing the first molecule's activity. For example, enzyme inhibitors are molecules that bind to enzymes thereby decreasing enzyme activity. The binding of an inhibitor may stop a substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both.

The terms “media composition”, “media preparation,” and “media formulation” are used interchangeably to refer to a media formulation that is able to maintain or sustain viability of one or a plurality of cells for at least a period of time. A media formulation can be complete or incomplete.

The term “complete media formulation” is used herein to refer to a mixture of components which, when used under appropriate conditions (e.g., at appropriate concentrations or dilutions, pH, temperature, % CO₂ or % O₂) are compatible with survival or proliferation of cells. Such media formulations are sufficient to maintain or sustain cell viability for at least a period of time, whether the cells proliferate or not, or whether the cells differentiate or not.

An “incomplete” media formulation typically lacks one or more components as compared to a complete media formulation, although lack of a particular component does not necessarily make an incomplete media formulation inadequate or insufficient to be compatible with survival or proliferation of cells.

The term “marker” is used herein to refer to a receptor, or a combination of receptors, found on the surface of a cell that allow a cell type to be distinguishable from other kinds of cells. Specialized protein receptors (markers) that have the capability of selectively binding or adhering to other signaling molecules coat the surface of every cell in the body. Cells use these receptors and the molecules that bind to them as a way of communicating with other cells and to carry out their proper function in the body.

TABLE 1
Markers Commonly Used to Identify Stem Cells and to
Characterize Differentiated Cell Types

| | | |
|---|---|---|
| Marker Name | Cell Type | Significance |

Blood Vessel

| | | |
|---|---|---|
| Fetal liver kinase-1 | Endothelial | Cell-surface receptor protein that identifies |
| (Flk1) | | endothelial cell progenitor; marker of cell- |
| | | cell contacts |
| Smooth muscle cell- | Smooth muscle | Identifies smooth muscle cells in the wall of |
| specific myosin heavy | | blood vessels |
| chain | | |
| Vascular endothelial cell | Smooth muscle | Identifies smooth muscle cells in the wall of |
| cadherin | | blood vessels |

Bone

| | | |
|---|---|---|
| Bone-specific alkaline | Osteoblast | Enzyme expressed in osteoblast; activity |
| phosphatase (BAP) | | indicates bone formation |
| Hydroxyapatite | Osteoblast | Mineralized bone matrix that provides |
| | | structural integrity; marker of bone |
| | | formation |
| Osteocalcin (OC) | Osteoblast | Mineral-binding protein uniquely |
| | | synthesized by osteoblast; marker of bone |
| | | formation |

Bone Marrow and Blood

| | | |
|---|---|---|
| Bone morphogenetic | Mesenchymal stem | Important for the differentiation of |
| protein receptor | and progenitor cells | committed mesenchymal cell types from |
| (BMPR) | | mesenchymal stem and progenitor cells; |
| | | BMPR identifies early mesenchymal |
| | | lineages (stem and progenitor cells) |
| CD4 and CD8 | White blood cell | Cell-surface protein markers specific for |
| | (WBC) | mature T lymphocyte (WBC subtype) |
| CD34 | Hematopoietic stem | Cell-surface protein on bone marrow cell, |
| | cell (HSC), satellite, | indicative of a HSC and endothelial |
| | endothelial | progenitor; CD34 also identifies muscle |
| | progenitor | satellite, a muscle stem cell |
| CD34 + Sca1+ | Mesenchymal stem | Identifies MSCs, which can differentiate into |
| Lin− profile | cell (MSC) | adipocyte, osteocyte, chondrocyte, and |
| | | myocyte |
| CD38 | Absent on HSC; | Cell-surface molecule that identifies WBC |
| | Present on WBC | lineages. Selection of CD34+/CD38- cells |
| | lineages | allows for purification of HSC populations |
| CD44 | Mesenchymal | A type of cell-adhesion molecule used to |
| | | identify specific types of mesenchymal cells |
| c-Kit | HSC, MSC | Cell-surface receptor on BM cell types that |
| | | identifies HSC and MSC; binding by fetal |
| | | calf serum (FCS) enhances proliferation of |
| | | ES cells, HSCs, MSCs, and hematopoietic |
| | | progenitor cells |
| Hoechst dye | Absent on HSC | Fluorescent dye that binds DNA; HSC |
| | | extrudes the dye and stains lightly |
| | | compared with other cell types |
| Leukocyte common | WBC | Cell-surface protein on WBC progenitor |
| antigen (CD45) | | |
| Lineage surface antigen | HSC, MSC | Thirteen to 14 different cell-surface proteins |
| (Lin) | Differentiated RBC | that are markers of mature blood cell |
| | and WBC lineages | lineages; detection of Lin-negative cells |
| | | assists in the purification of HSC and |
| | | hematopoietic progenitor populations |
| Mac-1 | WBC | Cell-surface protein specific for mature |
| | | granulocyte and macrophage (WBC |
| | | subtypes) |
| Muc-18 (CD146) | Bone marrow | Cell-surface protein (immunoglobulin |
| | fibroblasts, | superfamily) found on bone marrow |
| | endothelial | fibroblasts, which may be important in |
| | | hematopoiesis; a subpopulation of Muc-18+ |
| | | cells are mesenchymal precursors |
| Stem cell antigen | HSC, MSC | Cell-surface protein on bone marrow (BM) |
| (Sca-1) | | cell, indicative of HSC and MSC Bone |
| | | Marrow and Blood cont. |
| Stro-1 antigen | Stromal | Cell-surface glycoprotein on subsets of |
| | (mesenchymal) | bone marrow stromal (mesenchymal) cells; |
| | precursor cells, | selection of Stro-1+ cells assists in isolating |
| | hematopoietic cells | mesenchymal precursor cells, which are |
| | | multipotent cells that give rise to |
| | | adipocytes, osteocytes, smooth myocytes, |
| | | fibroblasts, chondrocytes, and blood cells |
| Thy-1 | HSC, MSC | Cell-surface protein; negative or low |
| | | detection is suggestive of HSC |

Cartilage

| | | |
|---|---|---|
| Collagen types II and IV | Chondrocyte | Structural proteins produced specifically by |
| | | chondrocyte |
| Keratin | Keratinocyte | Marker of epithelial differentiation |
| Sulfated proteoglycan | Chondrocyte | Molecule found in connective tissues; |
| | | synthesized by chondrocyte |

Fat

| | | |
|---|---|---|
| Adipocyte lipid-binding | Adipocyte | Lipid-binding protein located specifically in |
| protein (ALBP) | | adipocyte |
| Fatty acid transporter | Adipocyte | Transport molecule located specifically in |
| (FAT) | | adipocyte |
| Adipocyte lipid-binding | Adipocyte | Lipid-binding protein located specifically in |
| protein (ALBP) | | adipocyte |

Liver

| | | |
|---|---|---|
| Albumin | Hepatocyte | Principal protein produced by the liver; |
| | | indicates functioning of maturing and fully |
| | | differentiated hepatocytes |
| B-1 integrin | Hepatocyte | Cell-adhesion molecule important in cell-cell |
| | | interactions; marker expressed during |
| | | development of liver |

Nervous System

| | | |
|---|---|---|
| CD133 | Neural stem cell, | Cell-surface protein that identifies neural |
| | HSC | stem cells, which give rise to neurons and |
| | | gilial cells |
| Glial fibrillary acidic | Astrocyte | Protein specifically produced by astrocyte |
| protein (GFAP) | | |
| Microtubule-associated | Neuron | Dendrite-specific MAP protein found |
| protein-2 (MAP-2). | | specifically in dendritic branching of neuron |
| Myelin basic protein | Oligodendrocyte | Protein produced by mature |
| (MPB) | | oligodendrocytes; located in the myelin |
| | | sheath surrounding neuronal structures |
| Nestin | Neural progenitor | Intermediate filament structural protein |
| | | expressed in primitive neural tissue |
| Neural tubulin | Neuron | Important structural protein for neuron; |
| | | identifies differentiated neuron |
| Neurofilament (NF) | Neuron | Important structural protein for neuron; |
| | | identifies differentiated neuron |
| Neurosphere | Embryoid body (EB), | Cluster of primitive neural cells in culture of |
| | ES | differentiating ES cells; indicates presence |
| | | of early neurons and glia |
| Noggin | Neuron | A neuron-specific gene expressed during |
| | | the development of neurons |
| O4 | Oligodendrocyte | Cell-surface marker on immature, |
| | | developing oliciodendrocyte |
| O1 | Oligodendrocyte | Cell-surface marker that characterizes |
| | | mature oligodendrocyte |
| Synaptophysin | Neuron | Neuronal protein located in synapses; |
| | | indicates connections between neurons |
| Tau | Neuron | Type of MAP; helps maintain structure of |
| | | the axon. |

Pancreas

| | | |
|---|---|---|
| Cytokeratin 19 (CK19) | Pancreatic | CK19 identifies specific pancreatic epithelial |
| | epithelium | cells that are progenitors for islet cells and |
| | | ductal cells |
| Glucagon | Pancreatic islet | Expressed by alpha-islet cell of pancreas |
| Insulin | Pancreatic islet | Expressed by beta-islet cell of pancreas |
| Pancreas insulin- | Pancreatic islet | Transcription factor expressed by beta-islet |
| promoting factor-1 | | cell of pancreas |
| (PDX-1) | | |
| Nestin | Pancreatic | Structural filament protein indicative of |
| progenitor | progenitor | cell lines including pancreatic |
| Pancreatic polypeptide | Pancreatic islet | Expressed by gamma-islet cell of pancreas |
| Somatostatin | Pancreatic islet | Expressed by delta-islet cell of pancreas |

Pluripotent Stem Cells

| | | |
|---|---|---|
| Alkaline phosphatase | Embryonic stem | Elevated expression of this enzyme is |
| | (ES), embryonal | associated with undifferentiated pluripotent |
| | carcinoma (EC) | stem cell (PSC) |
| Alpha-fetoprotein | Endoderm | Protein expressed during development of |
| (AFP) | | primitive endoderm; reflects endodermal |
| | | differentiation Pluripotent Stem Cells |
| Bone morphogenetic | Mesoderm | Growth and differentiation factor expressed |
| protein-4 | | during early mesoderm formation and |
| | | differentiation |
| Brachyury | Mesoderm | Transcription factor important in the earliest |
| | | phases of mesoderm formation and |
| | | differentiation; used as the earliest indicator |
| | | of mesoderm formation |
| Cluster designation 30 | ES EC | Surface receptor molecule found |
| (CD30) | | specifically on PSC |
| Cripto (TDGF-1) | ES, cardiomyocyte | Gene for growth factor expressed by ES |
| | | cells, primitive ectoderm, and developing |
| | | cardiomyocytes |
| GATA-4 gene | Endoderm | Expression increases as ES differentiates |
| | | into endoderm. |
| GCTM-2 | ES EC | Antibody to a specific extracellular-matrix |
| | | molecule that is synthesized by |
| | | undifferentiated PSCs |
| Genesis | ES EC | Transcription factor uniquely expressed by |
| | | ES cells either in or during the |
| | | undifferentiated state of PSCs. |
| Germ cell nuclear factor | ES EC | Transcription factor expressed by PSCs |
| Hepatocyte nuclear | Endoderm | Transcription factor expressed early in |
| factor-4 (HNF-4) | | endoderm formation |
| Nestin | Ectoderm, neural | Intermediate filaments within cells; |
| | and pancreatic | characteristic of primitive neuroectoderm |
| | progenitor | formation |
| Neuronal cell-adhesion | Ectoderm | Cell-surface molecule that promotes cell- |
| molecule (N-CAM) | | cell interaction; indicates primitive |
| | | neuroectoderm formation |
| OCT4/POU5F1 | ES EC | Transcription factor unique to PSCs; |
| | | essential for establishment and |
| | | maintenance of undifferentiated PSCs |
| Pax6 | Ectoderm | Transcription factor expressed as ES cell |
| | | differentiates into neuroepithelium |
| Stage-specific embryonic | ES EC | Glycoprotein specifically expressed in early |
| antigen-3 (SSEA-3) | | embryonic development and by |
| | | undifferentiated PSC |
| Stage-specific embryonic | ES EC | Glycoprotein specifically expressed in early |
| antigen-4 (SSEA-4) | | embryonic development and by |
| | | undifferentiated PSCs |
| Stem cell factor (SCF | ES, EC, | Membrane protein that enhances |
| or c-Kit ligand) | HSC, MSC | proliferation of ES and EC cells, |
| | | hematopoietic stem cell (HSCs), and |
| | | mesenchymal stem cells (MSCs); binds the |
| | | receptor c-Kit |
| Telomerase | ES EC | An enzyme uniquely associated with |
| | | immortal cell lines; useful for identifying |
| | | undifferentiated PSCs |
| TRA-1-60 | ES EC | Antibody to a specific extracellular matrix |
| | | molecule is synthesized by undifferentiated |
| | | PSCs |
| TRA-1-81 | ES EC | Antibody to a specific extracellular matrix |
| | | molecule normally synthesized by |
| | | undifferentiated PSCs |
| Vimentin | Ectoderm, neural | Intermediate filaments within cells; |
| | and pancreatic | characteristic of primitive neuroectoderm |
| | progenitor | formation |

Skeletal Muscle/Cardiac/Smooth Muscle

| | | |
|---|---|---|
| MvoD and Pax7 | Myoblast, myocyte | Transcription factors that direct |
| | | differentiation of myoblasts into mature |
| | | myocytes |
| Myogenin and MR4 | Skeletal myocyte | Secondary transcription factors required for |
| | | differentiation of myoblasts from muscle |
| | | stem cells |
| Myosin heavy chain | Cardiomyocyte | A component of structural and contractile |
| | | protein found in cardiomyocytes |
| Myosin light chain | Skeletal myocyte | A component of structural and contractile |
| | | protein found in skeletal myocyte |

The term “mesenchymal cells (MSCs)” as used herein refers to adherent-capable multipotent stem cells displaying fibroblast-like morphology that differentiate from CFU-F cells present at low frequency in bone marrow, where they are immersed in the stroma (“marrow stromal cells”, “bone marrow stromal cells” and/or “stromal precursor cells”), are diversely distributed in several other tissues, and in ontogeny are capable of differentiating along several lineage pathways into osteoblasts, chondrocytes, myocytes and adipocytes. When referring to bone or cartilage, MSCs commonly are known as osteochondrogenic, osteogenic, chondrogenic, or osteoprogenitor cells, since a single MSC has shown the ability to differentiate into chondrocytes or osteoblasts, depending on the medium. MSCs secrete many biologically important molecules, including interleukins 6, 7, 8, 11, 12, 14, and 15, M-CSF, Flt-3 ligand, SCF, LIF, bFGF, VEGF, PIGF and MCP1.

The term “multipotent stem cells” as used herein refers to cells that can differentiate into multiple cell lineages, like osteoblast, chondroblast and adipocyte, but not all the lineages derived from the three germ layers. Examples include mesenchymal stem cells and several other adult stem cells.

Platelet derived growth factor (PDGF) is a major mitogen for connective tissue cells and certain other cell types. It is a dimeric molecule consisting of disulfide-bonded, structurally similar A and B-polypeptide chains, which combine to homo- and hetero-dimers. Activation of PDGF receptors leads to stimulation of cell growth, but also to changes in cell shape and motility; PDGF induces reorganization of the actin filament system and stimulates chemotaxis, i.e., a directed cell movement toward a gradient of PDGF.

The term “pluripotent stem cells” as used herein refers to cells that can differentiate into all the cells of the three embryonic germ layers forming the body organs, nervous system, skin, muscle and skeleton, but not embryonic components of the trophoblast and placenta. Examples include the inner cell mass of the blastocyst, embryonic stem cells, and reprogrammed cells, such as induced pluripotent stem (iPS) cells.

The term “progenitor cell” as used herein refers to an early descendant of a stem cell that can only differentiate, but can no longer renew itself.

The term “proliferation” as used herein refers to expansion of a population of cells by the continuous division of single cells into identical daughter cells.

The term “stem cells” refers to undifferentiated cells having high proliferative potential with the ability to self-renew (make more stem cells by cell division) that can generate daughter cells that can undergo terminal differentiation into more than one distinct cell phenotype

The term “supplement” as used herein refers to a component or ingredient that can be added to a complete or incomplete media formulation. Accordingly, a supplement of an incomplete media formulation can be a component of a complete media. For example, where an incomplete media lacks a medium component, a supplement for each such incomplete media can supply that missing medium component, and the resulting media then be considered a complete media.

Non-limiting examples of supplements include energy sources such as mono- or poly-saccharides (e.g., glucose or pyruvate); non-essential amino acids (e.g., alanine, asparagine, aspartate, glycine, proline or serine); hormones (e.g., insulin, insulin-like growth factor, a thyroid hormone such as thyroxine (T4) or triiodothyronine (T3), or a progesterone); cytokines and growth factors (e.g. epidermal growth factor (EGF), keratinocyte growth factor (KGF), hepatocyte growth factor (HGF), insulin like growth factor-1 and -2 (IGF-1, IGF-2), nerve growth factor (NGF)); interleukins and interferons; vitamins (e.g., A, B1, B2, B6 B12, C, D, E, K, biotin); heparin, heparin sulfate, buffers or salts (e.g., Earle's salts, Hanks' salts, Puck's salts, etc.), glycosaminoglycan degradation products, and co-factors. Additional supplements include, for example, β-mercaptoethanol, Leukemia Inhibitory Factor (LIF, ESGRO™), or serum substitutes, such as KNOCKOUT SR®, an FBS substitute for stem cell culture media.

Supplements also include, for example, animal sera, such as bovine sera (e.g., fetal bovine, newborn calf or normal calf sera) or human sera, typically at a concentration of about 1-25% (e.g., about 5-15%; about 10%); attachment factors or extracellular matrix components, such as collagens, laminins, proteoglycans, fibronectin, and vitronectin; and lipids, such as phospholipids, cholesterol, fatty acids, and sphingolipids.

Amounts or concentrations of a supplement can be determined by the particular media, growth conditions and cell types cultured in the media.

The terms “topically”, “topical administration” and “topically applying” are used interchangeably to refer to delivering a disclosed cosmetic composition o onto one or more surfaces of a tissue or cell, including epithelial surfaces. The composition may be applied by pouring, dropping, or spraying, if a liquid; rubbing on, if an ointment, lotion, cream, gel, or the like; dusting, if a powder; spraying, if a liquid or aerosol composition; or by any other appropriate means. Topical administration generally provides a local rather than a systemic effect.

The terms “VEGF-1” or “vascular endothelial growth factor-1” are used interchangeably herein to refer to a cytokine that mediates numerous functions of endothelial cells including proliferation, migration, invasion, survival, and permeability.

The term “without substantial differentiation,” when used in reference to stem cells, means that no more than about 20%, +/−5%, of the total number of stem cells in a given stem cell population have begun to differentiate or have differentiated. This term can be used to refer to one or a plurality of passages, e.g., 2, 3, 4, 5 or more passages, of a cell culture that includes stem cells.

The term “wrinkle” as used herein refers to a furrow, fold or crease in the skin.

### Biased Pluripotent Stem Cell Cultures

According to one aspect, disclosed herein are media formulations for preparing a biased stem cell population that secretes significant amounts of protein. According to some embodiments the media formulation is a chemically defined stem cell media. According to some embodiments, the media formulation is a chemically defined stem cell media comprising essential mineral nutrients, essential salts, essential amino acids, one or more supplements and hyaluronan. According to some embodiments, the media formulation at feeding comprises bFGF (10 ng/mL, 1-100 ng/mL) and activin A (5 ng/mL, 0.1-20 ng/mL) at feeding.

Exemplary formulations of a basal media and a protein supplement are reproduced in Table 2 and Table 3. Commercial basal media can be used in connection with the supplementation of Table 2. Exemplary commercial media include classic formulations such as DMEM, DMEM:F12, RPMI, and or modifications thereof.

TABLE 2
Basal media for stem cells

| Component Description | Mg/L |
|---|---|
| Calcium chloride anhydrous | 116.61 |
| Copper sulfate-5H₂O | 0.0013 |
| Potassium chloride | 312 |
| Magnesium chloride anhydrous | 28 |
| Magnesium sulfate anhydrous | 49 |
| Sodium chloride | 6250 |
| Sodium phosphate dibasic, anhydrous | 71 |
| Sodium phosphate monobasic H₂O | 62 |
| Zinc sulfate-7H₂O | 0.4 |
| L-Alanine | 9 |
| L-Arginine-HCl | 148 |
| L-Asparagine-H₂O | 16 |
| L-Aspartic acid | 20 |
| L-Cysteine-HCl—H₂O | 18 |
| L-Cystine-2HCl | 30 |
| L-Glutamic acid | 15 |
| Glycine | 20 |
| L-Histidine-HCl—H₂O | 30 |
| L-Isoleucine | 54 |
| L-Leucine | 59 |
| L-Lysine-HCl | 90 |
| L-Methionine | 17 |
| L-Phenylalanine | 35 |
| L-Proline | 20 |
| L-Serine | 30 |
| L-Threonine | 53 |
| L-Tryptophan | 9 |
| L-Tyrosine-2Na—2H₂O | 56 |
| L-Valine | 53 |
| Calcium D-pantothenate | 2.24 |
| Choline chloride | 9 |
| Folic acid | 2.5 |
| Myo-inositol | 12.6 |
| Niacinamide | 2 |
| Pyridoxal hydrochloride | 2 |
| Pyridoxine-HCl | 0.03 |
| Riboflavin | 0.22 |
| Thiamine-HCl | 2.17 |
| Vitamin B12 | 0.68 |
| D-Glucose | 2000 |
| HEPES | 3575 |
| Hypoxanthine-2Na | 2.7 |
| Linoleic acid | 0.04 |
| DL-Alpha-lipoic acid | 0.10 |
| Sodium pyruvate | 110 |
| Thymidine | 0.365 |
| Sodium bicarbonate | 2100 |

TABLE 3
10X Protein supplement for stem cells

| | Formulation (per 100 mL | |
|---|---|---|
| | supplement or 1L of final media) | |
| Components | Value | Unit |
| Water for injections | QS to 100 | ml |
| Human serum albumin | 3000 | mg |
| Transferrin, partially saturated | 20 | mg |
| Insulin | 20 | mg |
| T3 | 0.005 | mg |
| Selenite | 0.005 | mg |
| Taurine | 250 | mg |
| Hyaluronic acid | 0.1 | mg |
| Progesterone | 0.01 | mg |
| Vitronectin | 0.0025 | mg |
| Putrescine | 7.5 | mg |
| Glutathione, reduced | 0.5 | mg |
| Carnitine | 1 | mg |
| Ascorbyl phosphate | 75 | mg |
| Biotin | 50 | mg |
| L-glutamine | 365 | mg |
| HEPES | 1000 | mg |
| Ethanolamine | 15 | mg |

According to some embodiments, the supplement comprises albumin. According to some embodiments, the supplement comprises an iron carrier. Transferrin is an exemplary iron carrier. An iron carrier is typically a ligand for transferrin receptor. In some embodiments, the supplement is hyaluronic acid. Hyaluronic acid, a nonsulfated linear glycosaminoglycan (GAG), is a component of non-covalently formed complexes with proteoglycans in the extra-cellular matrix and is involved in the regulation of cell proliferation, adhesion and migration. Hyaluronic acid polymers are very large (with molecular weights of 100,000-10,000,000) and can displace a large volume of water. According to some embodiments, the supplement comprises glutamine.

The above media is also supplemented with bFGF (10 ng/mL, 1-100 ng/mL) and with activin A (5 ng/mL, 0.1-20 ng/mL) for feeding (maintenance of cultures). It is known that culture medium enriched with activin A, a pleiotropic cytokine that participates in developmental, inflammatory, and tissue repair processes, is capable of maintaining human embryonic stem cells in the undifferentiated state for more than 20 passages without the need for feeder layers, conditioned medium from mouse embryonic feeder layers, or STAT3 activation, and that the hESCs retain both normal karyotype and markers of undifferentiated cells, including Oct-4, nanog, and TRA-1-60, and remain pluripotent.

Biased stem cells are grown on an adherent substrate for the culture of pluripotent stem cells consisting of diluted MATRIGEL® (1:30 to 1:100) or a mixture of laminin and collagen or laminin and gelatin. The culture system is contained in tissue culture grade polystyrene vessels or bioreactors.

According to some embodiments, the cells are mammalian cells. According to some embodiments, the cells are ES cells. According to some embodiments, the cells are iPS cells. According to some embodiments, the cells are tissue-specific stem cells. According to some embodiments, the cells are germinal cells. According to some embodiments, the cells are adult stem cells. According to some embodiments, the cells are of human origin.

After a period of time, the cells are dissociated using collagenase IV (1-8 mg/mL). According to some embodiments, cells are dissociated when the density of the cells is at least 80%, at least 85%, at least 90%, at least 95%, or 100%. The cells are exposed for 5-10 min to the collagenase solution in a physiological buffer (e.g., Hanks Balanced Salt Solution (HBSS)). The collagenase solution is removed, the cells are lifted with a scraper in media and then dissociated by pipetting up and down with a serological pipette. According to some embodiments, the cell suspension is distributed into multiple or larger culture vessels at a proportion of 1:3, 1:4, 1:5, 1:6, 1:, 1:8, 1:9, or 1:10 of original surface to expand the cultures.

According to some embodiments, the activin A controls or reduces spontaneous differentiation in the cell cultures. Partially differentiated stem cells secrete multiple factors that can cause significant terminal differentiation. Folistatin has been identified as being secreted in high quantities in the biased cultures and is responsible for the differentiation of the stem cells. By adding activin A, the amount of spontaneous differentiation that is observed by a significant change of the cell morphology to an epithelial phenotype starting in the center of the denser colonies can be controlled. Uncontrolled, this phenomenon leads to complete differentiation of all pluripotent stem cells caused by continuous accumulation of folistatin.

According to some embodiments, partial blocking of endogenous follistatin signals (meaning preventing or obstructing by at least 90%, at least 85%, at least 80%, at least 75%, at least 70%, at least 65%, at least 60%, at least 55%, at least 50%, at least 45%, at least 40%, at least 35%, at least 30%, at least 25%, at least 20%, at least 15%, at least 20%, at least 5%) with activin A i results in a biased stem cell population that secretes significant amounts of protein.

Thus, a culture of biased stem cells comprises biased stem cells cultured in the presence of a growth medium, such as the medium of Table 2, supplemented with fetuin and activin A.

Exemplary biased stem cells useful in producing the biased stem cell cultures disclosed herein include, but are not limited to NIH nESC-14-0284, induced pluripotent stem cell lines, embryonic stem cell lines, and others.

The supernatant from biased stem cell cultures is collected using a defined schedule. An exemplary schedule is reproduced in Table 4.

TABLE 4
Collection schedule of the supernatant from biased stem cell cultures.

| | Day of Week | | | | | | |
|---|---|---|---|---|---|---|---|
| | Mon | Tues | Wed | Thurs | Fri | Sat | Sun |
| Feed/225 cm² | 50 | 60 | 100 | 50 | 60 | 100 | 100 |
| flask (mL) | | | | | | | |
| Passaged flasks | 8 (1:5) | — | — | 7 (1:6) | — | — | — |
| and ratio | | | | | | | |
| Collect/flask | 100 | 50 | 60 | 100 | 50 | 60 | 100 |
| (mL) | | | | | | | |
| Flasks in stock | 40 | 40 | 40 | 40 | 40 | 40 | 40 |
| Extra flasks | — | 32 | — | — | 33 | — | — |
| collected | | | | | | | |
| Total production | 4000 | 5200 | 2400 | 4000 | 5300 | 2400 | 4000 |
| (mL) | | | | | | | |

According to some embodiments, collecting is executed with sterile serological pipettes in sterile bottles or in plastic bags enclosed in a vacuum container.

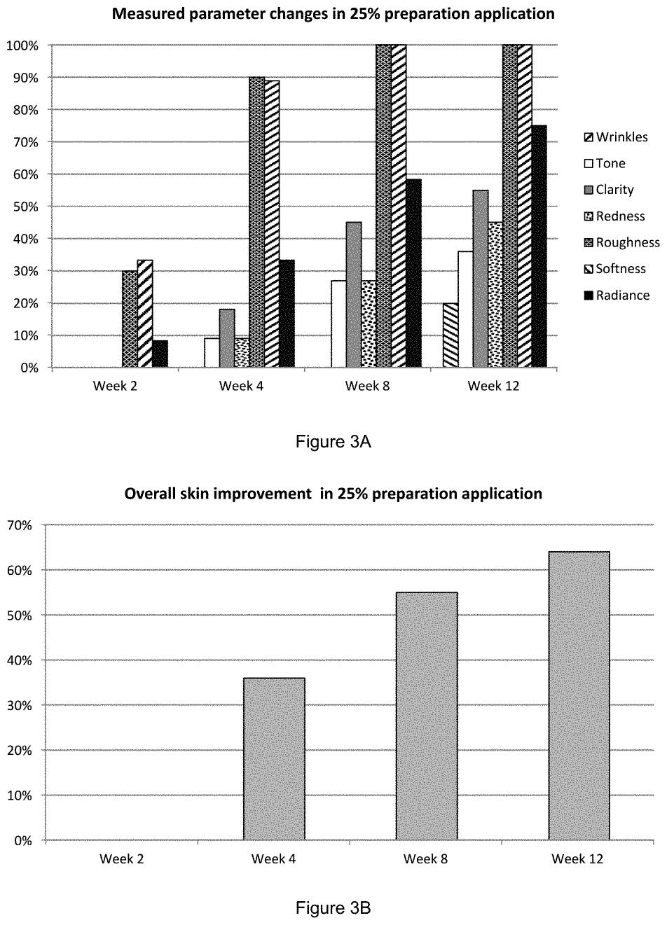

According to one embodiment, a method of collecting the supernatant from biased stem cell cultures is based on the principle of a lung box. An exemplary collection system is shown in FIG. 3. The system involves a plastic bag with a septum placed in a vacuum charged container. A needle connected to sterile tubing protruding in the infusion bag through the septum is used to aspirate the media from cultures or to connect to a bioreactor. A sterile tip can be used at the end of the tubing to ensure aseptic manipulation of the cultures.

The system consists of a rigid enclosure which withstands collapsing to about negative 30 psi. The box is provided with at least one wall made of transparent material, for example Lexan or glass with an anti-actinic coating to allow visual inspection of the bag and to protect the content against actinic radiations. The lid is attached with two hinges to the top of the box with non-protruding screws or with adhesive. The lid seals to the box enclosure with a rubber gasket. The lid has an opening larger than a standard infusion bag port diameter. A rubber plug for the opening in the lid is made of two halves with a center hole to accommodate the standard infusion bag tubing.

On the upper part of the box, a vacuum port is attached on one of the sides. The port can be provided with a valve or a quick-connect junction. The box can be vacuum pre-charged, not needing the attachment to a vacuum source. The collection system is assembled by placing the plastic infusion bag in the box and then leaving the tubing outside through the lid orifice. The two halves of the plug will be placed around the plastic bag tubing and inserted into the lid orifice. The aspiration tubing is inserted into the infusion bags port with a standard infusion needle, at the other end a clamping system applied to the tubing will provide the user control on the aspiration. A vacuum line carrying a negative pressure of −15 to −25 psi will be attached to the vacuum port. The media is collected by aspiration; filling the bag with air is avoided. If an important amount of air fills the bag, the vacuum can be discontinued, the box opened and positive pressure applied on the bag to eliminate the air. Alternatively the bag can have attached a second port with a vacuum line and clamp which can be activated to eliminate the excess air from the bag (evacuation port). A switch can control the vacuum to be applied to the bag or to the box. When the bag is full the system is disassembled and the bag removed from the box.

According to some embodiments, an industrial process of collecting supernatants from biased stem cell cultures comprises a reservoir of variable size connected anywhere in the system where a discard process is involved.

Following collection from two passages of biased stem cells as described in the collection schedule (Table 4), the media is refrigerated at 4° C. to 8° C. inclusive and remains stable for a time ranging from one hour to one month, e.g., at least 1 hr, at least 5 hr, at least 12 hr, at least 18 hr, at least 24 hr, at least 2 days, at least 3 days, at least 4 days, at least 5 days, at least 6 days, at least 7 days, at least 10 days, at least 2 weeks, at least 3 weeks, at least 4 weeks. The daily collections are pooled, mixed, and sterile filtered through a 0.22 pm or 0.1 pm low protein binding membrane, then distributed in sterile 100, 500 or 1000 mL bags or similar volume rigid containers.

According to some embodiments, the media collected from the biased cell culture contains a factor secreted by the biased cell culture into the media that is effective to modulate one or more of proliferation, inflammation, angiogenesis, and apoptosis.

According to some embodiments, the media collected from the biased cell culture contains an extracellular matrix factor secreted by the biased cell culture into the media.

According to some embodiments the media collected from the biased cell culture contains a growth factor secreted by the biased cell culture into the media. According to some embodiments, the growth factor has a stimulatory effect on cell proliferation. According to some embodiments, the growth factor has an inhibitory effect on cell proliferation. According to some embodiments, the growth factor has an anti-apoptotic effect on cells. According to some embodiments, the growth factor has a vasculogenic effect.

According to some embodiments, the media collected from the biased cell culture contains a cytokine secreted by the biased cell culture into the media. According to some embodiments, the cytokine has an inhibitory effect on the immune system. According to some embodiments, the cytokine has a stimulatory effect on the immune system.

According to some embodiments, the media collected from the biased cell culture contains a proteolytic enzyme secreted by the biased cell culture into the media.

According to some embodiments, the media collected from the biased cell culture contains an enzyme inhibitor secreted by the biased cell culture into the media.

### Cosmetic and Dermatological Compositions

Also disclosed herein are compositions comprising secretory products from cultures of biased pluripotent stem cells. According to some such embodiments the secretory products comprise human fetuin, wherein the fetuin is loaded or unloaded with stem cell-secreted factors.

Fetuin, or alpha 2HS glycoprotein, is a serum protein secreted mostly by liver and developing tissues, more abundant in the young organisms. The molecular structure allows a high affinity for calcium, with important role in preventing premature tissue calcification in young organisms. The calcium carrier property is crucial to re-establish local calcium homeostasis.

According to some embodiments, fetuin can be added in the culture from an external source (exogenous) or can be secreted by the biased pluripotent stem cells in culture (endogenous). According to some embodiments, the media collected from the biased pluripotent stem cell culture comprises fetuin. For example the fetuin secreted by embryonic stem cells will be different from the fetuin secreted by HepG2. Fetuin extracted from the serum of a young organism will be different from fetuin from an adult or aged organism because of the bound active molecules. According to some such embodiments, the cell culture characteristics will determine the physiological effect of the loaded fetuin. According to some embodiments, the fetuin derived from the biased pluripotent stem cell culture is loaded (meaning bound, complexed or otherwise associated) with active peptides and growth factors, as a signature of the originating tissue.

According to some embodiments, the fetuin derived from the biased pluripotent stem cell culture is loaded with at least some of the secreted products from the cultures of biased pluripotent stems cells.

According to some embodiments, the cosmetic compositions further comprise fatty acids, cholesterol or both. Because fetuin is 50 times more efficient in lipid transport than albumin, compositions containing fetuin and lipids have advantages over compositions containing albumin and lipids.

Fetuin in an aqueous solution can be mixed in various proportions with an amphoteric polymer, a polyelectrolyte, a surfactant or other protective compounds. A protective compound can be used to surround the fetuin molecules and provide cryptic protection, solubilization and chemical protection.

According to some embodiments, the human fetuin, factors bound to the human fetuin, or both can be released from the composition by an external stimuli, for example pH or temperature. For example, a temperature release based system is represented by an interpenetrating network of poly(acrylic acid) and polyacrylamide with 25° C. phase transition temperature; this system can become soluble upon heating or application on warm surface or skin.

In some embodiments, the cosmetic and dermatologic compositions comprise recombinant fetuin, in addition to, or instead of fetuin secreted by stem cells.

The cosmetic and dermatologic compositions disclosed herein can comprise one or more additional active ingredients, e.g., anti-microbial agents (e.g., benzoyl peroxyde, cetylpyridinium chloride, methylbenzethonium chloride, aluminium tris(hydroxy-benzenesulphonate), 6-chlorothymol, halocarban, 2,4-dichloro-3,5 xylenol, 3-amino-2-chlor-6-methylphenol, boric acid, [R—(Z)]-3-[(12-hydroxy-1-oxo-9-octadecenyl)amino] propyltrimethylammonium methyl sulphate, hexadecyltrimethyl-ammonium toluene-p-sulphonate retinol; antiviral agents (e.g., acyclovir); exfoliating agents (e.g., salicylic acid, urea); whitening agents (hydroquinone); therapeutic agents (e.g., contraceptives, nicotine, insulin, anti-cancer drugs etc.); or hormones.

According to some embodiments, the cosmetic and dermatologic compositions disclosed herein are effective to re-establish a normal epidermal calcium gradient. Mammalian epidermis displays a characteristic calcium gradient, with low calcium levels in the lower, basal, and spinous epidermal layers. Calcium levels increase progressively towards the outer stratum granulosum, and decline again in the stratum corneum.

Calcium concentration in the stratum corneum is very low in part because the relatively dry cells found in the stratum corneum are not able to retain the ions. Keratinocyte differentiation throughout the epidermis, is in part mediated by the calcium gradient. This calcium gradient parallels keratinocyte differentiation and as such is considered a key regulator in the formation of the epidermal layers. Low calcium concentrations stimulate proliferation of keratinocytes in the epidermis. High calcium concentrations inhibit their proliferation and enhance differentiation. Application of the described compositions to the top epidermal layers can reduce mineral precipitates in epidermis and prevent calcium accumulation across the entire epidermis as observed in aged skin. Fetuin can buffer mineral ion super-saturation and prevent unwanted calcium accumulation by formation of water-soluble fetuin-mineral complexes (FMC) or calciprotein particles (CPP).

According to some embodiments, the cosmetic and dermatologic compositions disclosed herein are effective to enhance the content of lipids of the epidermis. Increased incorporation of lipids in epidermis results in increased membrane resistance, increased cell volume and better water retention that translates overall to younger skin appearance. Fetuin is nearly 50 times more efficient than albumin as a carrier of free fatty acids. Application of the described compositions to the epidermis can result in increasing incorporation of exogenous fatty acids and increased triglyceride cell content, and the increased lipid content results in increased secretion of lipid lamellae (lamellar bodies) that enhances the skin barrier. Incorporation into lipids can be demonstrated in vitro on isolated keratinocyte culture and fibroblast culture.

According to some embodiments, cosmetic and dermatologic compositions disclosed herein, are effective to reduce scarring of epidermis. According to some such embodiments, the cosmetic compositions are effective to reduce scarring of the epidermis by blocking TGFβ. Fetuin directly binds to TGFβ and BMPs and thereby blocks binding of these factors to the extracellular domain of TGFβ-RII. Reducing the amount of TGFβ can prevent skin tumor formation. In addition will prevent scar formation by keloid fibroblast inhibition and ECM deposition.

Cosmetic and dermatologic formulations routinely are applied to the face and other areas of the skin and often remain on the skin for extended periods of time and are intended to beautify the wearer by providing color, contrast or otherwise changing or enhancing the appearance of the skin.

According to some embodiments, a cosmetic or dermatologic formulation prepared according to the present disclosure may take the compositional form of a liquid, a paste, a cream, a lotion, a powder, an ointment, or a gel.

According to some embodiments, the compositional form is a paste, meaning a semisolid dosage form that contains one or more substances intended for topical application.

According to some embodiments, the compositional form is a cream. The term “cream” as used herein refers to a viscous liquid or semisolid emulsion of either the oil-in-water or water-in-oil type. As used herein “emulsion” refers to a colloid system in which both the dispersed phase and the dispersion medium are immiscible liquids where the dispersed liquid is distributed in small globules throughout the body of the dispersion medium liquid. A stable basic emulsion contains at least the two liquids and an emulsifying agent. Common types of emulsions are oil-in-water, where oil is the dispersed liquid and an aqueous solution, such as water, is the dispersion medium, and water-in-oil, where, conversely, an aqueous solution is the dispersed phase. It also is possible to prepare emulsions that are nonaqueous. Creams of the oil-in-water type include hand creams and foundation creams. Water-in-oil creams include cold creams and emollient creams.

Creams may be diluted only with suitable diluents specified in the appropriate entries, and diluted creams must be freshly prepared without the application of heat.

According to some embodiments, the compositional form is a lotion, meaning a liquid or semi-liquid preparation that contains one or more active ingredients in an appropriate vehicle. A lotion may be a suspension of solids in an aqueous medium, an emulsion, or a solution. For example, according to some embodiments, the lotion is a shampoo or conditioner.

A “solution” generally is considered as a homogeneous mixture of two or more substances. It is frequently, though not necessarily, a liquid. In a solution, the molecules of the solute (or dissolved substance) are uniformly distributed among those of the solvent. Solvents that may be useful in the compositions of the present disclosure include water, as well as organic solvents, such as the alcohols.

According to some embodiments, the compositional form is a powder, also referred to as a dusting powder. The fineness of a powder often is expressed in terms of mesh size. Powders seeking to avoid any sensation of grittiness generally have a particle size of not more than 150 μm, i.e., less than 100-mesh.

According to some embodiments, the compositional form is an ointment. An ointment is a semi-solid preparation often intended for external application to the skin. Generally, ointment bases are categorized into hydrocarbon bases (oleaginous), adsorption bases (anhydrous); emulsion bases (water and oil type); and water soluble bases. Due to their anhydrous nature, ointments generally do not require any preservatives. They are more moisturizing and more occlusive than creams, and form a protective film over the skin. The occlusive effect tends to prolong and enhance penetration.

According to some embodiments, the compositional form of the present disclosure is a gel. The term “gel” as used herein refers to a sticky, jelly-like semisolid or solid prepared from high molecular weight polymers in an aqueous or alcoholic base. Alcoholic gels are drying and cooling, and are best suited for acute exudative pruritic eruptions; non-alcoholic gels are more lubricating and are well suited, for example, to dry scaling lesions.

Additional compositional forms may be prepared using technology readily known in the cosmetic arts, such as those described in Remington: The Science and Practice of Pharmacy, 20th Ed. (Gennaro, A. R. et al., eds) Lippincott Williams & Wilkins: Philadelphia (2000), which is incorporated herein by reference.

A number of additional ingredients can be added to the cosmetic and dermatologic compositions disclosed herein for functional, esthetic, and marketing purposes, including hydrophobic components, emulsifying agents, preservatives, humectants, thickeners, fragrances, dyes, pearlizers (e.g., bismuth oxychloride, BiOCl, which is a pearlescent pigment), herbal extracts, and vitamins, provided that the selected additional component(s) is chemically and physically compatible. The term “compatible” is used herein to mean that the components of the compositions are capable of being combined with each other in a manner such that there is no interaction that would substantially reduce the efficacy of the compositions under ordinary use conditions.

The cosmetic and dermatologic compositions disclosed herein can further include hydrophobic components which deliver skin conditioning benefits such as smoothness and softness to the skin as immediate perceivable effect along with the long term effect of fetuin. Exemplary hydrophobic components include, for example, fatty acids, silicone oils, mineral oil, petrolatum, C1-40 straight and branched hydrocarbons such as isohexadecane, C1-30 alcohol esters such as isopropyl isostearate, glycerides, alkylene glycol esters, propoxylated and ethoxylated derivatives, sugar esters such as sucrose polycottonseedate, vegetable oils such as coconut oil, hydrogenated vegetable oils, animal fats and oils, C4-20 alkyl ethers of polypropylene glycols, C1-20 carboxylic acid esters of polypropylene glycols, and di-C1-36 alkyl ethers.

Hydrophobic nonionic surfactants, which are surfactants that are water-insoluble and having an HLB value of less than 10, can be included as oily components. Exemplary hydrophobic nonionic surfactants include cetearyl glucoside, steareth-2, laureth-4, sucrose cocate, sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

According to some embodiments, the oily components are fatty alcohols, which provide skin conditioning benefits and can form gel networks with emulsifiers, which provide increased viscosity, phase stability, and conditioning benefits such as slippery feel. Exemplary fatty alcohols include saturated, linear or branched fatty alcohols, such as a saturated, linear or branched C₂₋₃₀ fMY-alcohols, saturated, linear or branched C₂₋₃₀ diols, and mixtures thereof. Further examples include cetyl alcohol, steacyl alcohol, and mixtures thereof.

According to some embodiments, the compositions comprise two or more oily components selected from the group consisting of hydrocarbon oils, fatty acid esters, and silicone oils.

The term “emollient” as used herein refers to fats or oils in a two-phase system (meaning one liquid is dispersed in the form of small droplets throughout another liquid). Emollients soften the skin by forming an occlusive oil film on the stratum corneum, preventing drying from evaporation in the deeper layers of skin. Thus, emollients are employed as protectives and as agents for softening the skin, rendering it more pliable. Emollients also serve as vehicles for delivery of hydrophobic compounds.

Exemplary emollients useful in the cosmetic and dermatologic compositions disclosed herein include, but are not limited to, wood alcohols, fatty alcohols (e.g., cetyl alcohol), propylene glycol, cocamidopropyl betaine, butylene glycol, pentylene glycol, ethylhexylglycerine, methoxy PEG-17, PEG-22/dodecyl glycol copolymers, alkylglucosides; butters, such as aloe butter, almond butter, avocado butter, cocoa butter, coffee butter, hemp seed butter, kokum butter, mango butter, mowrah butter, olive butter, sal butter, shea butter, glycerin, and oils, such as almond oil, aloe vera oil, apricot kernel oil, avocado oil, babassu oil, black cumin seed oil, borage seed oil, brazil nut oil, camellia oil, castor oil, coconut oil, emu oil, evening primrose seed oil, flaxseed oil, grape seed oil, hazelnut oil, hemp seed oil, jojoba oil, kukui nut oil, macadamia nut oil, meadowfoam seed oil, mineral oil, neem seed oil, olive oil, palm oil, palm kernel oil, peach kernel oil, peanut oil, plum kernel oil, pomegranate seed oil, poppy seed oil, pumpkin seed oil, rice bran oil, rosehip seed oil, safflower oil, sea buckthorn oil, sesame seed oil, shea nut oil, soybean oil, sunflower oil, tamanu oil, turkey red oil, walnut oil, or wheatgerm oil.

According to some embodiments, the oily components are included in the compositions at a level by weight of, for example about 2% to about 50%, about 2% to about 20%, or about 2% to about 10%, to provide skin conditioning benefits such as smoothness.

As used herein “emulsion” refers to a colloid system in which both the dispersed phase and the dispersion medium are immiscible liquids where the dispersed liquid is distributed in small globules throughout the body of the dispersion medium liquid. A stable basic emulsion contains at least the two liquids and an emulsifying agent.

According to some embodiments, an emulsifier is useful for dispersing the oil components in the aqueous phase. Exemplary emulsifiers include, without limitation, non-ionic and anionic emulsifiers, such as sugar esters and polyesters, alkoxylated sugar esters and polyesters, C₁-C₃₀ fatty acid esters of C₁-C₃₀ fatty alcohols, alkoxylated derivatives of C₁-C₃₀ fatty acid esters of C₁-C₃₀ fatty alcohols, alkoxylated ethers of C₁-C₃₀ fatty alcohols, polyglyceryl esters of C₁-C₃₀ fatty acids, C₁-C₃₀ esters of polyols, C₁-C₃₀ ethers of polyols, alkyl phosphates, polyoxyalkylene fatty ether phosphates, fatty acid amides, acyl lactylates, soaps, and mixtures thereof; polyethylene glycol 20 sorbitan monolaurate (polysorbate 20), polyethylene glycol 5 soya sterol, steareth-20, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, polyoxyethylene 20 sorbitan trioleate (polysorbate 85), sorbitan monolaurate, polyoxyethylene 4 lauryl ether sodium stearate, polyglyceryl-4 isostearate, hexyl laurate, PPG-2 methyl glucose ether distearate, ceteth-10, diethanolamine cetyl phosphate, glyceryl stearate, PEG 40 hydrogenated castor oil, PEG-60 hydrogenated castor oil, and mixtures thereof.

According to some embodiments, the present disclosure provides an oil-in-water composition comprising a microemulsion that is transparent or translucent in appearance. According to some such embodiments, the microemulsion can be formed via selection of surfactants. The surfactant selection for providing a microemulsion can be referred to as a “first surfactant system”. According to some embodiments, the first surfactant system is liquid at 40° C., more preferably at 25° C.

According to some embodiments, the first surfactant system comprises one or more nonionic surfactants, e.g., polysorbates, polyoxyalkylene hydrogenated caster oils, polyglycerin alkyl esters having the C₁₀₋₂₀ of alkylsubstitute, polyoxyethylene sterols, and polyoxyethylene hydrogenated sterols. According to some embodiments, the hydrophilic-lipophilic balance (HLB) of the first surfactant system as a whole is 10 or more, about 12 or more, or about 14 to about 20. According to some embodiments, the first surfactant system consists essentially of nonionic surfactants having an HLB of 10 or more, 12 or more, or from about 14 to about 20.

Exemplary polyoxyalkylene hydrogenated castor oils include polyoxyethylene hydrogenated castor oils having 20-100 moles of ethylene oxides, such as polyoxyethylene (20) hydrogenated castor oil, polyethylene (40) hydrogenated castor oil, and polyoxyethylene (100) hydrogenated castor oil.

Exemplary polyglycerin alkyl esters include those having 6-10 moles of glycerin units, such as polyglyceryl-6 laurate, polyglyceryl-10 laurate, and polyglyceryl-10 stearate.

Exemplary polysorbates include those having 20-80 moles of ethylene oxides, such as polysorbate-20, polyborbate-40, polysorbate-60, and polysorbate-80.

Exemplary polyethylene sterols and polyethylene hydrogenated sterols include those having 10-30 moles of ethylene oxides, such as polyethylene (10) phytosterol, polyethylene (30) phytosterol, and polyethylene (20) cholesterol.

According to some embodiments, the compositions comprises polysorbates, e.g., polysorbate-20, polysorbate-40, and mixtures thereof.

The term “carrier” as used herein refers to a cosmetically or dermatologically acceptable inert agent or vehicle for delivering one or more active agents to a subject, and often is referred to as “excipient.” The carrier must be of sufficiently high purity and of sufficiently low toxicity to render it suitable for administration to the subject being treated. The carrier further should maintain the stability and bioavailability of an active agent, e.g., a polypeptide disclosed herein. The carrier can be liquid or solid and is selected, with the planned manner of administration in mind, to provide for the desired bulk, consistency, etc., when combined with an active agent and other components of a given composition.

According to some embodiments, the described compositions comprise an aqueous carrier for providing the continuous phase. The level and species of the carrier are selected according to the compatibility with other components, and other desired characteristic of the product. The aqueous carrier is contained in the compositions at a level by weight of, for example, about 30% to about 98%, about 50% to about 95%, or about 70% to about 95%.

Exemplary carriers include water and water solutions of lower alkyl alcohols. Exemplary lower alkyl alcohols include monohydric alcohols having 1 to 6 carbons, e.g., ethanol. According to some embodiments, the aqueous carrier is substantially water.

The pH of the described compositions are, for example, about 4 to about 8 When skin benefiting agents are included in the compositions, the pH may be adjusted to that which provides optimum efficacy of the active skin benefiting agents. Buffers and other pH adjusting agents can be included to achieve the desirable pH. Exemplary pH adjusters herein include acetates, phosphates, citrates, triethanolamines and carbonates.

The viscosity (resistance to flow) of the described compositions may vary over a wide range, and may depend on viscosifying agents. For example, according to some embodiments, the described compositions may comprise a viscosifying agent that provides the compositions with a viscosity of from about 500 mPas to about 1,000,000 Pas. According to some embodiments, the viscosifying agent provides the compositions with a viscosisty of about 1,000 mPas to about 100,000 mPas.

Water-soluble or water-miscible viscosifying agents are those that are dissolved in a sufficient amount of water to result in a transparent solution.

Carboxylic acid/carboxylate copolymers are nonlimiting examplea of viscosifying agents used for providing microemulsions. Such copolymers can keep the composition relatively transparent and at a suitable viscosity without being tacky or greasy upon use, and can disperse and stabilize water insoluble components of the composition when such components are included. Exemplary commercially available carboxylic acid/carboxylate copolymers include acrylates/C₁₀₋₃₀ alkyl acrylate crosspolymers, e.g., PEMULEN™ TR-I, PEMULEN™ TR-2, CARBOPOL® 1342, CARBOPOL® 1382, and CARBOPOL® ETD 2020, all available from B. F. Goodrich Company.

Neutralizing agents, e.g., sodium hydroxide, potassium hydroxide, ammonium hydroxide, monoethanolamine, diethanolamine, triethanolamine, diisopropanolamine, am inomethylpropanol, tromethamine, tetrahydroxypropyl ethylenediamine, and mixtures thereof, may be included to neutralize the carboxylic acid/carboxylate copolymers.

Exemplary cellulose derivative polymers include, without limitation, methylcellulose, ethylcellulose, hydroxyethylcellulose, hydroxyethyl ethylcellulose, hydroxypropyl methyl cellulose, nitrocellulose, sodium cellulose sulfate, sodium carboxymethylcellulose, crystalline cellulose, cellulose powder, and mixtures thereof. According to some embodiments, the ceullulose derivative polymers Particularly preferred are hydroxyethylcellulose carboxymethylcellulose, and mixtures thereof. Commercially available compounds that are highly useful herein include hydroxyethylcellulose with tradename Natrosol Hydroxyethylcellulose, and carboxymethylcellulose with tradename Aqualon Cellulose Gum, both available from Aqualon.

Other exemplary viscosifying agents include pullulan, mannan, scleroglucans, polyvinylpyrrolidone, polyvinyl alcohol, guar gum, hydroxypropyl guar gum, xanthan gum, acacia gum, arabia gum, tragacanth, galactan, carob gum, karaya gum, locust bean gum, carrageenin, pectin, amylopectin, agar, quince seed (Cydonia oblonga Mill), starch (rice, corn, potato, wheat), and algae colloids (algae extract). Exemplary microbiological polymers include, without limitation, dextran, succinoglucan, starch-based polymers such as carboxymethyl starch, and methylhydroxypropyl starch. Exemplary alginic acid-based polymers include, without limitation, sodium alginate, and alginic acid propylene glycol esters. Exemplary acrylate polymers include, without limitation, sodium polyacrylate, polyacrylamide, and polyethyleneimine. Exemplary inorganic water soluble material includes, without limitation, bentonite, aluminum magnesium silicate, laponite, hectonite, and anhydrous silicic acid.

Polyalkylene glycols having a molecular weight of more than about 1000 also are exemplary viscosifying gents. Exemplary compounds include polyethylene oxides, polyoxyethylenes, and polyethylene glycols, polypropylene oxides, polyoxypropylenes, and polypropylene glycols; and polypropylene glycols and mixed polyethylene- polypropylene glycols, or polyoxyethylene-polyoxypropylene copolymer polymers. Exemplary polyethylene glycol polymers include, without limitation, PEG-2M, also known as POLYOX WSR® N-10, which is available from Union Carbide and available as PEG-2,000); PEG-5M, also known as POLYOX WSR® N-35; and POLYOX WSR® N-80, both available from Union Carbide and as PEG-5,000 and Polyethylene Glycol 300,000); PEG-7M, also known as POLYOX WSR® N-750 (available from Union Carbide); PEG-9M, also known as POLYOX WSR® N-3333 (available from Union Carbide); and PEG-14 M, also known as POLYOX WSR® N-3000 available from Union Carbide).

Exemplary commercially available additional water soluble polymers include, without limitation, xanthan gum (KELTROL™, available from Kelco), Carbomers (CARBOPOL™ 934, CARBOPOL™ 940, CARBOPOL™ 950, CARBOPOL™ 980, and CARBOPOL™ 981(all available from B. F. Goodrich Company), acrylates/steareth-20 methacrylate copolymer (ACRYSOL™ 22 (available from Rohm and Hass), polyacrylamide (SEPIGEL™ 305 (available from Seppic), glyceryl polymethacrylate (LUBRAGEL™ NP, and a mixture of glyceryl polymethacrylate, propylene glycol and PVM/MA copolymer (LUBRAGEL™ OIL (available from ISP), scleroglucan (CLEAROGEL™ SCI I available from Michel Mercier Products Inc. (NJ, USA)), ethylene oxide and/or propylene oxide based polymers (CARBOWAX™ PEGs, POLYOX™ WASRs, and UCON™ FLUIDS (all supplied by Amerchol).

Other exemplary agents include commercially available amphoteric polymers such as Polyquaternium 22 (MERQUAT™ 280, MERQUAT™ 295), Polyquaternium 39 (MERQUAT™ PLUS 3330, MERQUAT™ PLUS 3331), and Polyquaternium 47 (MERQUAT™ 2001, MERQUAT™ 200 IN), all available from Calgon Corporation.

The term “humectants” as used herein refers to substances that promote water retention due to their hygroscopicity. They act by being absorbed into the skin and attract water from the atmosphere. The attracted water then serves as a reservoir for the stratum corneum.

Exemplary water-soluble humectants include, without limitation, polyhydric alcohols, such as butylene glycol (1,3 butanediol), pentylene glycol (1,2-pentanediol), glycerin, sorbitol, propylene glycol, hexylene glycol, ethoxylated glucose, 1,2-hexane diol, 1,2-pentane diol, hexanetriol, dipropylene glycol, erythritol, trehalose, diglycerin, xylitol, maltitol, maltose, glucose, fructose; and other water-soluble compounds such as urea, sodium chondroitin sulfate, sodium hyaluronate, sodium adenosin phosphate, sodium lactate, pyrrolidone carbonate, glucosamine, cyclodextrin, and mixtures thereof. Additional examples include water soluble alkoxylated nonionic polymers such as polyethylene glycols and polypropylene glycols of molecular weight of up to about 1000 (e.g., PEG-200, PEG-400, PEG-600, PEG-1000), and mixtures thereof.

Commercially available humectants include, without limitation: butylene glycol (1,3-Butylene glycol, available from Celanese), pentylene glycol (HYDROLITE™-5 available from Dragoco), glycerin (STAR™ and SUPEROL™, available from The Procter & Gamble Company, CRODEROL™ GA7000 available from Croda Universal Ltd., PRECERIN™ series available from Unichema, and a same tradename as the chemical name available from NOF; propylene glycol (LEXOL™ PG-865/855 available from Inolex, 1,2-PROPYLENE GLYCOL USP available from BASF; sorbitol (LIPONIC™ series available from Lipo, SORBO™, ALEX™, A-625™, and A-641™ available from ICI, and UNISWEET™ 70, UNISWEET™ CONC available from UPI; dipropylene glycol with the same tradename available from BASF; diglycerin (DIGLYCEROL™, available from Solvay GmbH); xylitol with the same tradename available from Kyowa and Eizai; maltitol (MALBIT™ available from Hayashibara; sodium chondroitin sulfate with the same tradename available from Freeman and Bioiberica, and with tradename ATOMERGIC SODIUM CHONDROITIN SULFATE available from Atomergic Chemetals; sodium hyaluronate, available from Chisso Corp. the same with tradenames ACTIMOIST™ available from Active Organics, AVIAN SODIUM HYALURONATE series, available from Intergen, HYALURONIC ACID Na, available from Ichimaru Pharcos; sodium adenosine phophate with the same tradename available from Asahikasei, Kyowa, and Daiichi Seiyaku; sodium lactate with the same tradename available from Merck, Wako, and Showa Kako, cyclodextrin (CAVITRON™ available from American Maize, RHODOCAP™ series available from Rhone-Poulenc, and DEXPEARL™ available from Tomen); polyethylene glycols (CARBOWAX™ series available from Union Carbide), and a mixture of glyceryl polymethacrylate, propylene glycol and PVM/MA copolymer (LUBRAJEL™ Oil available from Guardian Lab).

The term “preservative” is used herein to refer to substances that prevent the growth of undesired microorganisms in products that contain water. Preservatives approved for use in cosmetics may be identified in the current Federal Regulations published in volume 21 of the Code of Federal Regulations, which is incorporated herein by reference. Exemplary preservatives include, without limitation: ascorbic acid, ascorbyl palmitate, biopein, BHT (butylated hydroxyl-toluene), butylated hydroxyanisole, butylated hydroxytoluene, butylparaben, calcium ascorbate, calcium sorbate, citric acid, cinnamon cassia, chlorocresol, diazolidinyl urea, dilauryl thiodipropionate, EDTA (ethylenediamine tetraacetic acid tetrasodium salt), erythorbic acid, grapefruit seed extract, hydroxyhenzoates, methylparaben, Neopein, phenonip, phenoxyethanol, potassium bisulfite, potassium metabisulfite, potassium sorbate, propylparaben, rosemary oil extract, sodium ascorbate, sodium benzoate, sodium bisulfite, sodium metabisulfite, sodium sorbate, sodium sulfite, sorbic acid, sulfur dioxide, Suprarein, thiodipropionic acid, and/or tocopherols.

The described compositions may further comprise a safe and effective amount of an additional skin active agent. Exemplary skin active agents include, without limitation, skin lightening agents, anti-acne agents, emollients, non-steroidal anti-inflammatory agents, topical anaesthetics, artificial tanning agents, antiseptics, anti-microbial and anti-fungal actives, skin soothing agents, sunscreening agents, skin barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, and mixtures thereof. When included, the present compositions comprise from about 0.001% to about 30%, preferably from about 0.001% to about 10% of an additional skin active agent.

The type and amount of skin active agents are selected so that the inclusion of a specific agent does not affect the stability of the composition.

Skin lightening agents are active ingredients that improve hyperpigmentation as compared to pre-treatment. Exemplary skin lightening agents include, without limitation, ascorbic acid compounds, azelaic acid, butyl hydroxyanisole, gallic acid and its derivatives, glycyrrhizinic acid, hydroquinone, kojic acid, arbutin, mulberry extract, and mixtures thereof. Combinations of skin lightening agents may be advantageous in that they may provide skin lightening benefit through different mechanisms.

Exemplary ascorbic acid compounds include, without limitation, ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein include, for example, esters of ascorbic acid, and ester salts of ascorbic acid. According to some embodiments, the ascorbic acid compounds include 2-o-D-glucopyranosyl-L-ascorbic acid, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-o-D-glucopyranosyl-L-ascorbic acid available from Hayashibara and sodium L-ascorbyl phosphate (STAY™ C50 available from DSM).

Other exemplary hydrophobic skin lightening agents include, without limitation, ascorbic acid derivatives such as ascorbyl tetraisopalmitate (for example, VC-IP available from Nikko Chemical), ascorbyl palmitate (for example available from DSM), ascorbyl dipalmitate (for example, NIKKOL CP available from Nikko Chemical); undecylenoyl phenyl alanine (for example, SEPIWHITE MSH available from Seppic); octadecenedioic acid (for example, ARLATONE DIOIC DCA available from Uniquema); Oenothera biennis sead extract, and pyrus malus (apple) fruit extract, and mixtures thereof.

Additional skin active agents include, without limitation, panthenol, benzoyl peroxide, 3-hydroxy benzoic acid, farnesol, phytantriol, glycolic acid, lactic acid, 4-hydroxybenzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoicacid, tocopherol and its esters (e.g., tocopherol acetate), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2,-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

The described compositions may further comprise a safe and effective amount of a UV absorbing agent. Exemplary UV protecting agents include, without limitation, those described in U.S. Pat. Nos. 5,087,445, 5,073,372, 5,073,371,; and Segarin, et al, at Chapter VIII, pages 189 et seq., of Cosmetics Science and Technology (1972). When included, in the disclosed compositions, the U.V. absorbing agent comprises from about 0.5% to about 20%, preferably from about 1% to about 15% of the described composition by weight.

Exemplary UV absorbing agents include 2-ethylhexyl-p-methoxycinnamate (commercially available as PARSOL™ MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzim idazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-{4-methylbenzylidene) camphor, EUSOLEX™ 6300, Octocrylene, Avobenzone (commercially available as PARSOL 1789), and mixtures thereof.

The described compositions may further contain additional components such as are conventionally used in topical products, e.g., for providing aesthetic or functional benefit to the composition or skin, such as sensory benefits relating to appearance, smell, or feel, therapeutic benefits, or prophylactic benefits. It is to be understood that the above-described required materials may themselves provide such benefits.

Exemplary topical ingredient classes include: anti-cellulite agents, antioxidants, radical scavengers, chelating agents, vitamins and derivatives thereof, abrasives, other oil absorbents, astringents, dyes, essential oils, fragrance, structuring agents, emulsifiers, solubilizing agents, anti-caking agents, antifoaming agents, binders, buffering agents, bulking agents, denaturants, pH adjusters, propellants, reducing agents, sequestrants, cosmetic biocides, and preservatives.

Many references exist for teaching how to create acceptable formulations of cosmetics and dermatologic products, including Handbook of Cosmetic Science and Technology, Second Edition, Marc Paye, et al. (Editor), CRC Press (2006), which is expressly incorporated herein by reference.

For example, facial foundation is used to make the skin look natural and beautiful for as long as possible. To do so, it unifies the color of the skin, improve a dull and tired complexion, give a matte finish, and masks possible imperfections, e.g., dark spots, small wrinkles, dark rings under the eye, and the pores of the skin surface. Its application must be easy and give coverage for a natural complexion, it must have a pleasant texture, a good adhesive property, be comfortable, and have a consistent color and smooth finish. Thus, provided herein are facial foundations comprising the cosmetic compositions disclosed herein.

[symbol]

For example, formulations for face foundations can be in liquid, gel, cream, solid cream, cake, mousse or stick form. There are four basic facial foundation formulations: oil-in-water, water-in-oil, oil-free, and water-free or anhydrous forms.

For example, oil-based foundations are water-in-oil emulsions containing pigments suspended in oil, e.g., mineral oil. The formulation may include vegetable oils (e.g., coconut and sesame) and synthetic esters (octyl palmitate and isopropyl myristate). Oil-based formulations also contain, e.g., water, silicone tension-actives, vitamins, UV filters, moisturizing agents, etc.

Oil-free foundations can contain vegetable oils, mineral oils, and other oily substances (e.g., silicones dimethicone or cyclomethicone, which leave the skin with a dry feeling). They come in three forms: alcohol based, glycerine based, and creams or lotions. The smooth feeling of a foundation depends on the physical properties of the raw material pigments, such as particle size, shape, etc.

Face powders provide coverage of complexion imperfections, oil control, a matte finish and tactile smoothness to the skin. Exemplary ingredients include talc and serecite (to help to spread), chalk or kaolin (to give moisture-absorbing qualities), magnesium stearate (for adherence), zinc oxide and titanium oxide, and pigments. Mica improves skin feel, product application and skin adhesion. Mica can be modified by coating with inorganic or organic materials to produce another group of fillers (spherical, special, and surface modified). Spherical fillers are used to improve skin feel. Examples of organic spherical fillers include polyacrylamides, and nylon spheres; examples of inorganic spherical fillers include silica, both as solid or hollow spheres. When spherical materials are used, there is an increase in the viscosity of the emulsion, allowing for a reduction of viscosity modifiers in the final formulation. Special fillers are a group of fillers made into a composite material. For example, mica can be coated with very small particles of metal oxides, allowing ease of incorporation into liquid formulations. Examples of coating materials for micas are titanium dioxide, barium sulfate, BiOCI, and organic compounds. For surface modified fillers, exemplary coating materials are organic polymers, e.g., collagen, elastin and vitamin E. Powders also can contain organic texture agents (polymers) or mineral agents (boron nitride and silica), preservatives, antioxidants and perfumes. Thus, disclosed herein are facial powders comprising the cosmetic compositions disclosed herein.

### EXAMPLES

The following examples are put forth so as to provide those of ordinary skill in the art with a complete disclosure and description of how to make and use the presently compositions, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Identification and Quantification of Proteins in the Biased Stem Cell Culture Supernatant

Stem cell line NIH hESC-14-0284 (see National Institutes of Health Human Embryonic Stem Cell Registry) was expanded using the media formulation and methods provided herein. The supernatant was collected daily and pooled over one week or two passages of cell culture to ensure a homogenization (meaning a blending of unlike elements In order to distribute them equally throughout) of the factors secreted at various cell densities during expansion. The samples were filtered through a 0.1 μm pore PVDF filter and frozen in 1 mL aliquots at −20° C. until analysis.

Samples of the media were analyzed using a quantitative ELISA assay using antibodies specific to each identified protein. The assay was performed using a QUANTI BODY® assay (RayBiotech). QUANTIBODY® is an array-based multiplex ELISA system for simultaneous quantitative measurement of multiple cytokines and growth factors.

An exemplary list of factors secreted by the stem cultures and identified by the QUANTI BODY® method (within or above the linear range of assay) is shown in Table 5.

TABLE 5
Exemplary list of factors secreted by biased stem cell culture.

| | | Maximum | | |
|---|---|---|---|---|
| | | Linear | | |
| | Level of | Assay | Detected | |
| | Detection | Value | Value | |
| Target | (pg/ml) | (pg/mL) | (pg/mL) | Description |
| A2M | 44.1 | 40,000 | 3,757 | alpha-2-Macroglobulin |
| ACE-2 | 926.6 | 400,000 | 3,637 | Angiotensin-converting enzyme 2 |
| Adipsin | 24.2 | 20,000 | 177 | Factor D |
| AFP | 21.1 | 10,000 | 2,211 | Alpha fetal protein |
| Albumin | 26.6 | 20,000 | 39,394 | Principal serum protein |
| ALCAM | 14.4 | 10 000 | 46.1 | Activated leukocyte cell adhesion |
| | | | | molecule |
| ANG | 1.4 | 2,000 | 506 | Angiogenin |
| ANG-1 | 49.3 | 40,000 | 439 | Angiopoietin 1 |
| ANG-2 | 27.1 | 20,000 | 85.9 | Angiopoietin 2 |
| ANGPTL4 | 122.7 | 400,000 | 4,303 | Angiopoietin-like 4e |
| ApoA1 | 270.6 | 100,000 | 2,448 | Apolipoprotein A-1 |
| ApoC1 | 5.4 | 10,000 | 3,966 | Apolipoprotein C1 |
| ApoC2 | 309.6 | 200,000 | 23,758 | Apolipoprotein 02 |
| ApoC3 | 0.6 | 2,000 | 701 | Apolipoprotein 03 |
| ApoE | 42.0 | 40,000 | 14,928 | Apolipoprotein E |
| ApoH | 170.1 | 100,000 | 21,873 | Apolipoprotein H |
| Artemin | 8.9 | 10,000 | 80.6 | Neurotrophin in the glial cell line- |
| | | | | derived neurotophic factor (GDNF) |
| | | | | family |
| B2M | 14.1 | 10,000 | 7,904 | β2 microglobulin |
| bFGF | 33.1 | 20,000 | 25,114 | Basic fibroblast growth factor |
| bIG-H3 | 36.5 | 10 000 | 12,005.3 | Transforming growth factor, beta- |
| | | | | induced |
| CEA | 434.7 | 20,000 | 2,330.3 | Carcinoembryonic antigen |
| Chemerin | 123.5 | 66 667 | 3,375.6 | Retinoic acid receptor responder |
| | | | | protein 2 |
| CHI3L1 | 10.1 | 10,000 | 31.4 | Chitinase-3-like protein 1 |
| Clusterin | 21.5 | 10,000 | 36,364.0 | |
| CRP | 34.6 | 10,000 | 333.7 | C-reactive protein |
| CRTAM | 13.7 | 4,000 | 195.2 | Cytotoxic and regulatory T cell |
| | | | | molecule |
| CTLA4 | 24.6 | 4,000 | 159.7 | Cytotoxic T-Lymphocyte Antigen 4, |
| | | | | CD152 |
| CXCL16 | 11.4 | 20,000 | 496.7 | Chemokine for CTL pro-inflammatory |
| Cystatin C | 220.1 | 100,000 | 6,178.7 | |
| Decorin | 3.9 | 2,000 | 3,837.2 | Pericellular matrix proteoglycan |
| Dkk-3 | 35.8 | 100,000 | 1,725.5 | Dickkopf-related protein 3 |
| Dkk-4 | 88.2 | 100,000 | 588.3 | Dickkopt-related protein 4 |
| EMMPRIN | 2.7 | 2,000 | 331.3 | Extracellular matrix metalloproteinase |
| | | | | inducer CD147 |
| ENA-78 | 7.7 | 10,000 | 2,275.1 | CXCL5 |
| Endostatin | 6.4 | 10,000 | 1,170.4 | |
| Ferritin | 9739.2 | 800,000 | 62,184.1 | |
| Fetuin A | 97.1 | 100,000 | 589,999.6 | Fetal type glycoprotein |
| FGF-19 | 16.5 | 20,000 | 2,031.6 | Fibroblast growth factor 19 |
| Fibrinogen | 42.3 | 40,000 | 196.3 | Plasma protein |
| Follistatin | 44.7 | 40,000 | 34,502.3 | |
| FSH | 24.6 | 10,000 | 179.9 | Follicle stimulating hormone. |
| Galectin-3 | 8.7 | 4,000 | 138.4 | |
| Gas1 | 305.4 | 100,000 | 15,329.9 | Growth arrest-specific 1 |
| GASP-1 | 4.5 | 2,000 | 249.6 | Growth and differentiation factor |
| | | | | (GDF)-associated serum protein-1 |
| GDF-15 | 1.4 | 2,000 | 547.8 | TGFb family member |
| GRO | 4.0 | 1,000 | 108.3 | CXCL1 |
| GROa | 2436.3 | 100,000 | 91,979.5 | Neutrophil chemoattractant activity |
| CXCL1 | | | | |
| HAI-2 | 38.4 | 40,000 | 443.7 | Hepatocyte growth factor activation |
| | | | | inhibitor 2 |
| HCC-1 | 5.8 | 1,333 | 53.2 | CCl-14 |
| hCGb | 38.9 | 20,000 | 11,075.5 | Human chorionic gonadotropin-beta |
| Hemo-globin | 306.0 | 100,000 | 9,977.4 | erythrocyte protein |
| HGF | 7.2 | 4,000 | 365.4 | Hepatocyte growth factor |
| ICAM-2 | 294.7 | 100,000 | 6,380.5 | Intercellular adhesion molecule 2 |
| IGFBP-1 | 15.3 | 5,000 | 52.7 | Insulin-like growth factor binding |
| | | | | protein 1 |
| IGFBP-2 | 66.6 | 20,000 | 38,569.7 | Insulin-like growth factor binding |
| | | | | protein 2 |
| IGFBP-3 | 161.2 | 200,000 | 11,461.6 | Insulin-like growth factor binding |
| | | | | protein 3 |
| IGFBP-4 | 910.7 | 200,000 | 3,652.8 | Insulin-like growth factor binding |
| | | | | protein 4 |
| IGFBP-6 | 115.0 | 100,000 | 4,499.0 | Insulin-like growth factor binding |
| | | | | protein 6 |
| IL-21 | 703.3 | 100,000 | 5,817.7 | Interleukin 21 |
| IL-23 | 102.2 | 40,000 | 516.7 | Interleukin 23 |
| IL-27 | 7.4 | 10,000 | 183.3 | Interleukin 27 |
| Insulin | 50.2 | 20,000 | 25,280.3 | |
| LAP | 17.0 | 4,000 | 472.3 | Latency Associated Peptide |
| Legumain | 26.2 | 10,000 | 3,204.1 | |
| Lipocalin- | 3.3 | 1,000 | 298.9 | Oncogene 24p3 or neutrophil |
| 2 | | | | gelatinase-associated lipocalin |
| L-Selectin | 301.8 | 100,000 | 5,580.8 | CD62L |
| MCP-1 | 4.4 | 2,000 | 1,109.5 | CCL2, monocyte chemotactic protein 1 |
| MICB | 25.6 | 15,000 | 127.9 | Ligand for the NKG2D type II receptor. |
| MIF | 3.9 | 4,000 | 658.0 | Macrophage migration inhibitory factor. |
| MMP-10 | 8.3 | 10,000 | 31.9 | Metalloproteinase 10 |
| MMP-2 | 394.4 | 100,000 | 1,264.6 | Metalloproteinase 2 |
| MMP-9 | 12.0 | 20,000 | 362.3 | Metalloproteinase 9 |
| Nidogen-1 | 47.9 | 20,000 | 34,800.9 | Entactin |
| NOV | 9.4 | 4,000 | 759.8 | ECM-associated signaling protein. |
| NSE | 182.0 | 100,000 | 4,466.1 | Neuron specific enolase |
| OPN | 207.3 | 100,000 | 12,498.8 | osteopontin |
| PAI-I | 137.1 | 40,000 | 31,246.4 | Plasminogen activator inhibitor-1 |
| PARC | 16.9 | 4,000 | 91.8 | p53-associated Parkin-like cytoplasmic |
| | | | | protein |
| P- | 128.1 | 100,000 | 46,218.0 | placental cadherin |
| Cadherin | | | | |
| PDGF-AA | 3.6 | 10,000 | 388.9 | Platelet derived growth factor AA |
| Pepsinogen I | 9.2 | 20,000 | 2,685.5 | |
| Periostin | 37.2 | 200,000 | 237.0 | Ligand for alpha-V/beta-3 and alpha- |
| | | | | V/beta-5 integrins |
| PF4 | 222.1 | 100,000 | 4,529.4 | Platelet factor 4 |
| PGRP-5 | 0.8 | 1,000 | 10.6 | Mammalian peptidoglycan |
| | | | | recognition protein |
| PIGF | 7.4 | 4,000 | 47.5 | |
| RBP4 | 16.2 | 20,000 | 26,858.6 | Retinol binding protein 4 |
| Serpin A4 | 10.2 | 10,000 | 5,434.2 | Serpin peptidase inhibitor, clade A |
| | | | | member 4 |
| sFRP-3 | 76.9 | 100,000 | 3,375.7 | Secreted frizzled-related protein 3 |
| TFPI | 42.5 | 100,000 | 12,757.1 | Tissue factor pathway inhibitor |
| TIMP-1 | 41.8 | 40,000 | 31,787.7 | Metallo-protease inhibitor 1 |
| TIMP-2 | 98.1 | 40,000 | 69,788.6 | Metallo-protease inhibitor 2 |
| TIMP-4 | 14.6 | 20,000 | 400.5 | Metalloproteinase inhibitor 4 |
| tPA | 3.9 | 1,000 | 434.8 | Tissue plasminogen activator |
| Trans-ferrin | 124.0 | 100,000 | 122,227.7 | Plasma iron carrier |
| Trappin-2 | 18.8 | 10,000 | 312.0 | Serine protease inhibitor |
| TSP-1 | 178.2 | 100,000 | 29,268.2 | Extracellular matrix |
| TSP-2 | 24.8 | 10,000 | 14,173.8 | Extracellular matrix |
| uPA | 2.3 | 1,333 | 723.9 | Plasminogen activator- urokinase |
| VE- | 601.4 | 200,000 | 333,881.8 | Extracellular matrix |
| Cadherin | | | | |
| VEGF | 11.3 | 10,000 | 149.3 | Vascular endothelial growth factor |
| Vitronectin | 94.1 | 100,000 | 320.7 | Extracellular matrix |
| vWF | 95.9 | 100,000 | 6,470.0 | Von Willebrand factor |
| WIF-1 | 48.9 | 20,000 | 193.0 | Wnt inhibitor factor |

The majority of the growth factors, cytokines, and hormones detected and retrieved in sub-physiological quantities are related to cell survival, migration and proliferation. There are abundant extracellular circulating proteins (such as albumin, fetuin, apolipoproteins, transferrin, fibrinogen) and matrix proteins (cadherins, proteoglicans) that constitute carriers of growth factors, hormones and cytokines.

There is a marked difference in the growth factor spectrum reported by others in stem cell supernatants, which likely is a consequence of the differences in the culture conditions used.

### Example 2

### Clinical and Histologic Evaluation of Skin Exposed to Stem Cell Conditioned Media

Skin aging is a cumulative and multi-factorial process in which both intrinsic and extrinsic determinants lead progressively to a loss of structural integrity and physiological function of the skin. For example, as skin ages, cell renewal can decrease dramatically so skin looks dry and dull.

Intrinsic aging of the skin occurs as a natural consequence of physiological changes over time at variable genetically determined rates. Extrinsic aging is mediated by environmental factors, including, without limitation, exposure to sunlight, pollution, nicotine, repetitive muscle movements, such as squinting or frowning, and miscellaneous lifestyle components, such as diet, sleeping position and overall health.

Youthful skin is characterized by its unblemished, evenly pigmented, smooth, pink appearance.

Intrinsically aged skin is comparatively thin, inelastic and finely wrinkled with deepening of facial expression lines. These changes are evidenced histologically as a thinned epidermis and dermis with flattening of the rete pegs at the dermo-epidermal junction (DEJ, the intersection of the epidermis and dermis).

Cutaneous aging due to sun exposure is known as photoaging. The rate of change in the skin due to photoaging is dependent on many intrinsic and extrinsic or environmental factors, including, without limitation, genetic background of the individual, environmental latitude at which sun exposure takes place, intensity and duration of sun exposure in outdoor activities of sport, employment or leisure, and to some extent, vigor of prevention or treatment. Extrinsically aged, sun-exposed skin appears clinically as blemished, thickened, yellowed, lax, rough, and leathery. These changes may begin as early as the second decade. As used herein, the term “photo-damaged” when referring to skin includes skin damaged and other causes of skin damage.

Irregular hyperpigmentation and hypopigmentation, both discrete and limited or diffuse and irregular may be noted, and clinically represented by freckles, solar lentigines (blemishes on the skin that range in color from light brown to red or black), and hypomelanotic macules (meaning a flat, distinct, colored area of skin that is less than 1 centimeter in diameter and does not include a change in skin texture or thickness). An appearance and feel of surface roughness, dryness or scaliness may be partially explained by abnormalities of keratinocyte production, adhesion and separation. Wrinkles of various depth, length and location are a reflection of underlying dermal damage to collagen, elastin and ground substance and their incomplete repair. Orientation of deeper wrinkles according to lines of underlying muscular forces may be pronounced. The color of photo-aged skin may be sallow in some instances but otherwise is variable due to the irregularity of surface and of reflected light as well as to the variability of total skin thickness, melanin content and distribution, and influence of saturated and unsaturated hemoglobin.

Photo-aged skin is characterized histologically by epidermal dysplasia with varying degrees of cytologic atypia, loss of keratinocyte polarity, an inflammatory infiltrate, decreased collagen, increased ground substance and elastosis. Elastosis (accumulation of amorphous elastin material) is characteristic of photo-aged skin. By light microscopy, three different types of fibers are observed in normal human skin: oxytalan, elaunin and elastic fibers. Elaunin fibers are a component of elastic fibers formed from a deposition of elastin between oxytalan fibers. Elastic fibers consisting of microfibrils and an amorphous substance containing elastin, branch and anastomose to form networks and fuse to form fenestrated membranes and elastic laminae. Oxytalan fibers, the most superficial ones, which are located in the papillary dermis are very thin, directed perpendicularly to the dermatoepidermal junction, and are formed by bundles of tubular microfibrils 10 to 12 nm in diameter. UV exposure induces a thickening and coiling of elastic fibers in the papillary dermis and, with chronic UV exposure, in the reticular dermis. UV-exposed skin manifests a reduction in the number of microfibrils and increases in interfibrillar areas. The initial response of elastic fibers to photo-damage is hyperplastic (meaning an abnormal multiplication of cells), resulting in a greater amount of elastic tissue. The level of sun exposure determines the magnitude of the hyperplastic response. In aged elastic fibers, a secondary response to photo-damage occurs but is degenerative, with a decrease observed in skin elasticity and resiliency. In aged elastic fibers, a secondary response to photo-damage occurs but is degenerative, with a decrease observed in skin elasticity and resiliency.

Photo-aged skin also may accumulate changes to epidermal cell DNA and result in many benign and malignant neoplasms of the skin. These include benign seborrheic keratosis (round or oval skin growths that originate in keratinocytes that appear in various colors from light tan to black), actinic keratosis (a premalignant condition of thick, scaly or crusty patches of skin) and squamous cell carcinoma. Some of the propensity toward cancerous growths may be due to a decrease in Langerhans cells and their function. Aged skin may also contain telangiectasias (small dilated blood vessels) and lentigines (blemishes on the skin that range in color from light brown to red or black).

The following study was performed to examine the effect of stem cell conditioned media on photo-aged skin.

Stem cell line NIH hESC-14-0284 was expanded using the media formulation and methods provided herein. The supernatant was collected daily and pooled over one week (two passages of cell culture) to ensure a homogenization (meaning a blending of unlike elements In order to distribute them equally throughout) of the factors secreted at various cell densities during expansion. The samples were filtered through a 0.1 μm pore PVDF filter and frozen in 50 mL aliquots at −20° C. until use.

A sample preparation incorporating 5% or 25% of conditioned media in 1,2-Pentanediol (HYDROLITE®5, Symrise) and MIKROKILL™ COS (Arch Personal Care) was used to demonstrate: 1) histologic evidence of increases in fibrillin, collagen 1-3, elastin, and other histologic markers as determined from a 3 mm punch biopsy obtained from the sun exposed outer arm; and 2) improvement in the appearance of facial lines/wrinkles, firmness/elasticity (e.g., where firmness refers to the range in appearance from loose, sagging skin to firm skin), radiance (where radiance refers to the range in appearance from dull/flat looking skin to bright and luminous skin), skin texture/smoothness (e.g., where texture refers to the range in appearance from smooth skin to rough skin), and overall appearance based on subject efficacy questionnaires.

Eight female subjects (4 subjects applied composition with 25% conditioned media, 4 subjects applied composition with 5% conditioned media) 40-59 years of age of any race or skin type with baseline visual analog scale (VAS) lines/wrinkles greater than or equal to 2 and baseline VAS elasticity greater than or equal to 3.5, were enrolled in this single site multiple dilution pilot study to evaluate the effect of the stem cell conditioned media preparation on facial photoaging.

VAS yields a numerical assessment value which can be evaluated for subjective characteristics or attitudes that cannot be directly measured. When responding to an item in a VAS scale, an evaluator specifies his/her level of agreement to a statement by indicating a position along a line (10 cm) between two end-points or anchor responses.

Subjects who signed an informed consent form and met all inclusion criteria were asked to use only DOVE® bar soap and no other facial moisturizers or cleansers. Subjects were then asked to apply the composition over the entire face and the entire upper left arm from the elbow to the shoulder, morning and evening for a total of 12 weeks. Both the investigator and the subject assessed the appearance of the facial skin in terms of lines/wrinkles, firmness/elasticity, radiance, skin texture/smoothness, and overall appearance based on efficacy questionnaires. Photographs of the central, right, and left face were taken. Subjects returned to the office at week 2 for questionnaire completion and photography. Additional visits were conducted at week 4 and week 8 where the identical activities were performed. At week 12, in addition to questionnaires and photography, one biopsy was taken from the upper outer left arm, which was treated, and one biopsy was taken from the upper outer right arm, which was untreated, and both biopsy sites were closed with one suture. The study concluded at week 14 with removal of the sutures.

### Inclusion Criteria

Subject with Baseline VAS lines/wrinkles AND Baseline VAS elasticity/firmness≥3.5;

Subject agrees not to introduce any new makeup/cosmetics, toiletries or personal care products, other than the provided test material, during the course of the study;

Subject has signed an Informed Consent Form in compliance with 21 CFR Part 50: “Protection of Human Subjects”;

Subject is dependable and able to follow directions and is willing to comply with the schedule of visits;

Subject is in generally good health.

### Exclusion Criteria

Subject is pregnant or nursing;

Subject has received treatment with sympathomimetics, antihistamines, vasoconstrictors, non-steroidal anti-inflammatory agents, and/or systemic or topical corticosteroids within one week prior to initiation of the study;

Subject has a history of acute or chronic dermatologic, medical, physical conditions, which would preclude application of the test material and/or could influence the outcome of the study;

Concomitant medication restriction. All oral medications remained unchanged during the 12-week study. No topical medications were allowed on the face or left arm during the study. Only DOVE® soap can be used on the face or arm.

### Conduct of Study: Methods and Procedures

A signed informed consent form was obtained from each subject before performing any study procedures. No study related procedures or activities were performed until each subject was fully informed and the consent form was signed and dated.

An abbreviated medical history including current medications were recorded. All subjects were healthy with an unremarkable medical history.

A dermatological examination was performed. Patients had no skin conditions that might interfere with the study results in the opinion of the dermatologist investigator. The investigator excluded subjects from participation based on the examination results or other concerns.

The subjects were screened for the inclusion and exclusion criteria prior to study enrollment. Only subjects who met the requirements, signed an informed consent form, and gave a medical history were entered into the study. All other subjects were considered screening failures.

All subjects were randomized to receive either the 5% or the 25% stem cell conditioned media concentration. Randomization was based on the subject number that was assigned based on the order in which the subjects presented to the research center for enrollment. 4 subjects were placed in each concentration groups.

The study subjects were capable of applying stem cell conditioned media preparation to the entire face and the left arm from the elbow to the shoulder twice daily for 12 weeks. The subjects applied the product twice daily to the entire face and the left arm from the elbow and the upper arm.

### Clinical Evaluations

Subjects were evaluated by collecting a variety of observations beginning at washout and continuing through visits at baseline, week 2, week 4, week 8, and week 12.

At each visit the following were performed: a subjective questionnaire, a visual grading by both subjects and investigator as to lines/wrinkles, tone, clarity, redness, texture/smoothness, softness, radiance/luminosity, and overall appearance. A full face photograph and photographs of each side of the face were taken. At week 12 a punch biopsy was taken (3 mm on the sun exposed left treated arm, 3 mm on the sun exposed right untreated arm).

The evaluation was performed using a five-point ordinal scale (0-4): 0=none; 1=minimal; 2=mild; 3=moderate; 4=severe

A study termination form was completed for each study subject who received study product. This included subjects who completed the study or who withdrew or were withdrawn from study. 8/8 subjects successfully completed the study per protocol.

No adverse events or adverse experiences occurred during the administration of this study.

### Results

Investigator Longitudinal Analysis with Percent Change from Baseline. The investigator-assessed improvement in all subjects using the study product beginning at week 2. Results are shown in Table 6. Statistically significant improvement was seen in tactile skin roughness (p=0.043). This improvement continued into week 4 where statistically significant improvement was seen in tactile roughness (p=0.001), visual softness (p=0.002), and light reflected radiance (p=0.004). Overall the skin was improved in a highly statistically significant fashion (p=0.001). By week 8, all parameters except wrinkles and redness had improved in a statistically significant fashion. Continuing improvement was see at week 12, where all parameters were statistically significant to include wrinkles (p=0.004), skin tone (e.g., ranging from uneven to even) (p=0.003), clarity (e.g., ranging from blotchy to clear) (p=0.006), redness (p=0.016), roughness (e.g., ranging from tactilely rough to tactilely smooth) (p<0.001), softness (p<0.001), radiance (e.g., ranging from dull/flat looking skin to bright and luminous skin) (p=0.001), and overall appearance (p<0.001).

TABLE 6
Longitudinal analysis performed
by investigator compared to baseline

| | | | Observed | Baseline | |
|---|---|---|---|---|---|
| | Time | | Average | average | p |
| Analysis | (week) | Criteria | score | score | (t test) |
| 25% preparation | 4 | Roughness | 0.250 | 2.500 | 0.027 |
| against baseline | 4 | Softness | 0.250 | 2.250 | 0.036 |
| | 4 | Overall | 1.750 | 2.750 | 0.044 |
| | 8 | Tone | 2.000 | 2.750 | 0.05 |
| | 8 | Clarity | 1.500 | 2.750 | 0.035 |
| | 8 | Softness | 0.000 | 2.250 | 0.015 |
| | 8 | Radiance | 1.250 | 3.000 | 0.026 |
| | 8 | Overall | 1.250 | 2.750 | 0.024 |
| | 12 | Tone | 1.750 | 2.750 | 0.044 |
| | 12 | Clarity | 1.250 | 2.750 | 0.036 |
| | 12 | Roughness | 0.000 | 2.500 | 0.015 |
| | 12 | Softness | 0.000 | 2.250 | 0.015 |
| | 12 | Radiance | 0.750 | 3.000 | 0.018 |
| | 12 | Overall | 1.000 | 2.750 | 0.013 |
| 5% preparation | 4 | Roughness | 0.500 | 2.500 | 0.019 |
| against baseline | 4 | Softness | 0.500 | 2.500 | 0.019 |
| | 4 | Radiance | 1.750 | 3.250 | 0.016 |
| | 4 | Overall | 1.750 | 3.250 | 0.016 |
| | 8 | Tone | 2.250 | 3.250 | 0.044 |
| | 8 | Roughness | 0.000 | 2.500 | 0.014 |
| | 8 | Softness | 0.000 | 2.500 | 0.014 |
| | 8 | Radiance | 1.500 | 3.250 | 0.017 |
| | 8 | Overall | 1.250 | 3.250 | 0.016 |
| | 12 | Tone | 1.500 | 3.250 | 0.044 |
| | 12 | Redness | 0.500 | 2.000 | 0.038 |
| | 12 | Roughness | 0.000 | 2.500 | 0.014 |
| | 12 | Softness | 0.000 | 2.500 | 0.014 |
| | 12 | Radiance | 1.250 | 3.250 | 0.016 |
| | 12 | Overall | 1.000 | 3.250 | 0.013 |

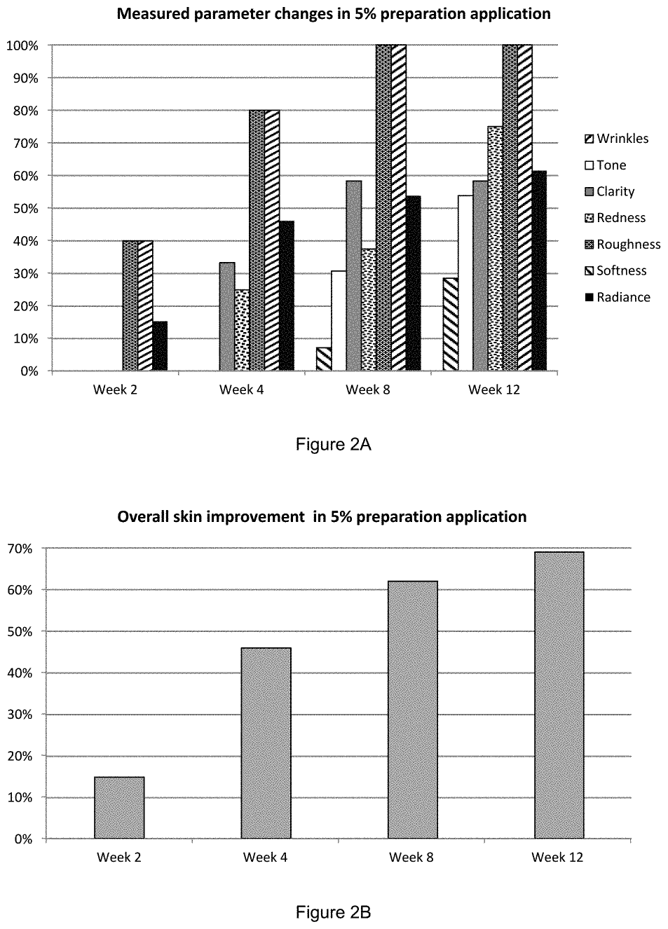

FIGS. 2A and 3A show measured parameter changes with application of the 5% preparation and 25% preparation, respectively. FIGS. 2B and 3B show overall skin improvement with application of the 5% and 25% preparation, respectively, with percent change from baseline.

The subjects rated a statistically significant improvement in softness at week 12 (p=0.042). All parameters were improved at week 12 in the percent improvement analysis (wrinkles reduced 11%, tone improved 13%, clarity improved 18%, redness reduced 29%, roughness reduced 20%, softness improved 40%, radiance improved 18%, and overall appearance improved 11%) indicating a favorable response.

### Histological Analysis

Punch biopsies from the treated area of the arm and an identical non-treated area from the contralateral arm were fixed in formaldehyde, sectioned and stained with hematoxylin and eosin for morphological features (nuclear counts, rete pegs) and labeled antibodies against aquaporin 3, filaggrin, and element collagen.

Aquaporin 3 is a membrane transporter of water and glycerol expressed in plasma membranes in the basal layer keratinocytes of epidermis in normal skin, whose expression increases in response to skin stress.

Filaggrin is a structural protein in the skin that facilitates the compaction of keratinocytes and promotes the formation of the stratum corneum. During epidermal terminal differentiation, the ˜400 kDa profilaggrin polyprotein is dephosphorylated and rapidly cleaved by serine proteases to form monomeric filaggrin (37 kDa), which binds to and condenses the keratin cytoskeleton, contributing to the cell compaction process that produces the squamous cell phenotype of the stratum corneum. In the stratum corneum, filaggrin is further processed into hygroscopic small molecular weight molecules such as urea and amino acids, collectively referred to as natural moisturizing factor. Loss of profilaggrin or filaggrin leads to a poorly formed stratum corneum (ichthyosis), which is also prone to water loss (xerosis).

To avoid minor local particularities, the whole slide digital scans were used for the evaluation and quantification.

Rete pegs are undulating ridges, which increase surface area between the epidermal and dermal skin layers. The presence of rete pegs improves skin integrity and strength. As skin ages the presence of rete pegs decreases, resulting in a flattening of the dermo-epidermal junction, accompanied by a thinning of the epidermis and dermis.

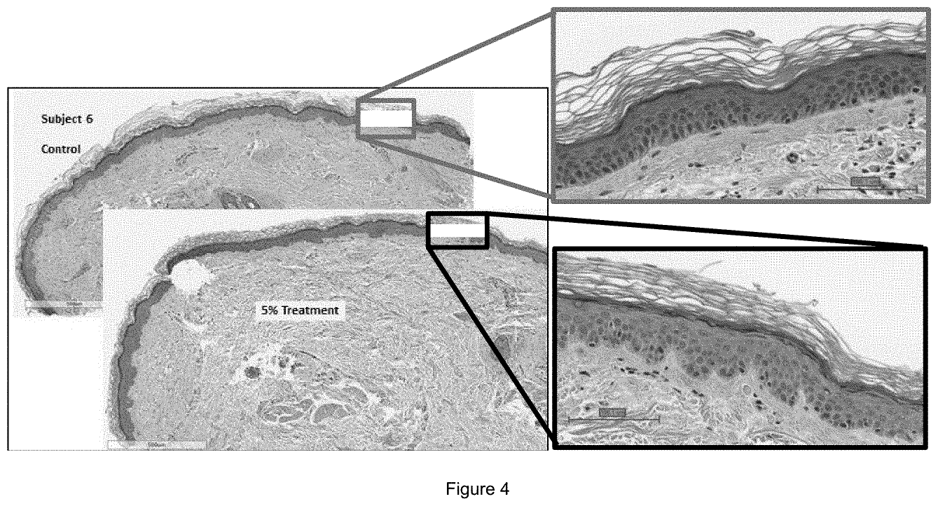

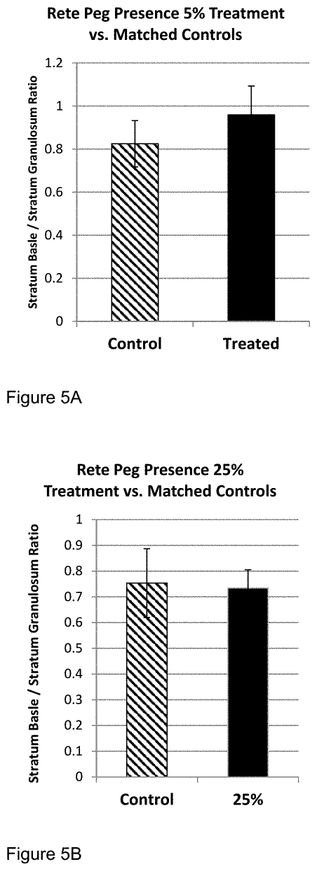

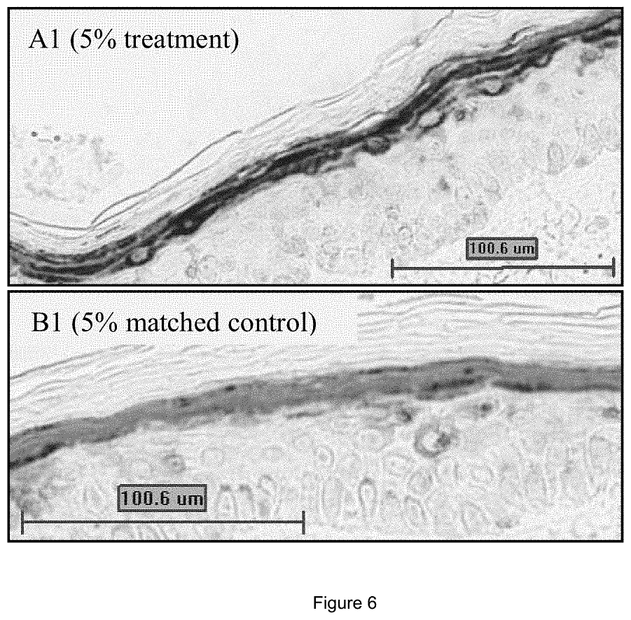

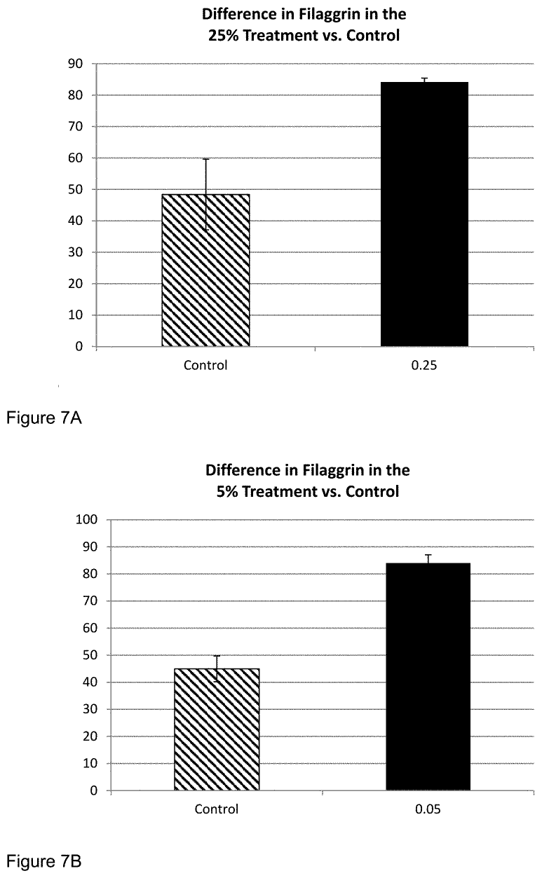

As shown in FIGS. 4 and 5, the presence of rete pegs is visibly increased after treatment with the disclosed cosmetic composition. FIG. 6 shows representative images of filaggrin immunochemistry. The positive staining shows increased intensity in the treatment sample. As shown in FIG. 7, histological comparison of biopsies from treated subjects and matched controls showed that filaggrin levels increased significantly (p<0.001) in the treatment arm vs. matched controls.

No statistical significance was found between nuclear counts of the control and treated area (p=0.150) or for collagen and aquaporin 3 content of the treated and non-treated area (p>0.1).

### Summary

Safety assessment: No adverse events were reported by either the investigator or the subjects.

Tolerability Assessment: There was no observed or reported irritation after 12 weeks of using the study product.

Histologic markers as determined from a 3 mm punch biopsy obtained from the sun exposed outer arm suggest increased barrier function and overall appearance by significant increase of rete pegs and filaggrin.

Self-assessment by subjects demonstrate improvement in lines/wrinkles, firmness/elasticity, radiance, skin texture/smoothness, and overall appearance. The self-assessment was confirmed by the dermatologist assessment.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A cosmetic or dermatologic composition comprising:
a) a culture media supernatant collected from a culture comprising a population of biased pluripotent stem cells being characterized by expression of stem cell markers OCT4 and SSEA4, wherein the biased pluripotent stem cells are biased by culturing the pluripotent stem cells without a feeder layer in a cell culture basal medium with a supplement comprising taurine, ethanolamine, biotin, ascorbyl phosphate, hyaluronic acid, glutamine, basic fibroblast growth factor (bFGF), and activin A, wherein the culture media supernatant comprises secretory products of the population of biased pluripotent stem cells and human fetuin; and
b) at least one cosmetically or dermatologically acceptable carrier, and
i) an effective amount of at least one further ingredient selected from a hydrophobic component, an emulsifier, a water-soluble humectant, an emollient, a viscosifying agent, an ultraviolet absorbing agent, or an additional skin active agent; or
ii) the composition is in form of a microemulsion. wherein the composition comprises about 0.001 μg/ml to about 1 μg/ml of human fetuin, and
wherein an effective amount of the cosmetic or dermatologic composition is effective to enhance appearance of skin.

2. The cosmetic or dermatologic composition according to claim 1, wherein the fetuin is secreted by the biased pluripotent stem cells.

3. The cosmetic or dermatologic composition according to claim 1, wherein the effective amount of the composition is effective to enhance appearance of skin by improving one or more of tactile roughness, visual softness, light reflected radiance, appearance of lines/wrinkles, skin tone, skin clarity, redness, firmness/elasticity, radiance, skin texture/smoothness, or overall appearance.

4. The cosmetic or dermatologic composition according to claim 1, wherein the effective amount of the secretory product of the population of biased pluripotent stem cells is effective to modulate one or more of proliferation, inflammation, angiogenesis, or apoptosis of epidermal cells.

5. The cosmetic or dermatologic composition according to claim 1, wherein the secretory product of the population of biased pluripotent stem cells includes:
(a) an effective amount of an extracellular matrix factor secreted by the biased cell culture into the culture media; or
(b) an effective amount of a growth factor secreted by the biased cell culture into the culture media; or
(c) a combination thereof.

6. The cosmetic or dermatologic composition according to claim 5, wherein the growth factor is a cytokine.

7. The cosmetic or dermatologic composition according to claim 1, wherein the secretory product of the population of biased pluripotent stem cells includes:
(a) an effective amount of a proteolytic enzyme secreted by the biased cell culture into the culture media; or
(b) an effective amount of an enzyme inhibitor secreted by the biased cell culture into the culture media; or
(c) a combination thereof.

8. The cosmetic or dermatologic composition according to claim 1, wherein the population of biased pluripotent stem cells is of human origin.

9. The cosmetic or dermatologic composition according to claim 1, wherein the composition comprises an effective amount of at least one further ingredient selected from a hydrophobic component, an emulsifier, a water-soluble humectant, a viscosifying agent, a ultraviolet absorbing agent; or an additional skin active agent.

10. The cosmetic or dermatologic composition according to claim 1, further comprising pentylene glycol.

11. The cosmetic or dermatologic composition according to claim 1, wherein the composition is in form of a microemulsion.

12. The cosmetic or dermatologic composition according to claim 1, wherein the skin is photo-damaged skin.

13. The cosmetic or dermatologic composition according to claim 1, wherein the skin is aged skin.

14. The cosmetic or dermatologic composition according to claim 1, wherein the culture media is present in the cosmetic composition at a concentration of about 1% to about 25%.

15. The cosmetic or dermatologic composition according to claim 1, wherein the culture media is present in the cosmetic composition at a concentration of 1%, 2%, 3%, 4%, 5%, 10%, 15%, 20%, or 25%.

16. The cosmetic or dermatologic composition according to claim 1, wherein the population of biased pluripotent stem cells comprises a population of induced pluripotent stem (iPS) cells, a population of embryonic stem (ES) cells, a population of germinal cells, a population of tissue-specific stem cells, or a population of adult stem cells.

17. The cosmetic or dermatologic composition according to claim 1, comprising exogenous human fetuin.

18. The cosmetic or dermatologic composition according to claim 1, further comprising an iron carrier.

19. The cosmetic or dermatologic composition according to claim 35, wherein the iron carrier in the basal medium protein supplement comprises transferrin.

20. The cosmetic or dermatologic composition according to claim 1, wherein the fetuin secreted into the culture media supernatant is present in a concentration from at least 0.001 μg/ml to about 1 μg/ml.

21. A cosmetic or dermatologic composition comprising:
an effective amount of human fetuin and at least one cosmetically or dermatologically acceptable carrier,
wherein an effective amount of the cosmetic or dermatologic composition is effective to enhance appearance of skin.

22. A method for enhancing appearance of skin, comprising
(a) providing a cosmetic or dermatologic composition according to claims 1;
(b) applying an effective amount of the composition topically; and
(c) improving one or more of tactile roughness, visual softness; light reflected radiance; appearance of lines/wrinkles, skin tone; skin clarity, redness; firmness/elasticity, radiance, skin texture/smoothness, or overall appearance.
